SEQUENCE LISTING (47 sequences)

SEQ ID NO: 1 - DNA, 73 bp, Artificial Sequence
  Synthetic oligonucleotide
cgtgacatagctatcaactcgctctcagtacaatataatgctatgacatcttagctaaccactatactagatg

SEQ ID NO: 2 - DNA, 73 bp, Artificial Sequence
  Synthetic oligonucleotide
uatutagtatagtggttagataagatgtuataguattatattgtautgagagugagttgatagutatgtuaug

SEQ ID NO: 3 - DNA, 73 bp, Artificial Sequence
  Synthetic oligonucleotide
ugtgauatagutatuaautugututuagtauaatataatgutatgauatuttagutaauuautatautagatg

SEQ ID NO: 4 - DNA, 24 bp, Artificial Sequence
  Synthetic oligonucleotide
aaacaaattaataactatatcaca

SEQ ID NO: 5 - DNA, 73 bp, Artificial Sequence
  Synthetic oligonucleotide
catctaatataataattaactaaaatatcataacattatattatactaaaaacaaattaataactatatcaca

SEQ ID NO: 6 - DNA, 24 bp, Artificial Sequence
  Synthetic oligonucleotide
tttagttaactattatattagatg

SEQ ID NO: 7 - DNA, 73 bp, Artificial Sequence
  Synthetic oligonucleotide
catctaatataatagttaactaaaatatcataacattatattatactaaaaacaaattaataactatatcaca

SEQ ID NO: 8 - DNA, 73 bp, Artificial Sequence
  Synthetic oligonucleotide
tgtgatatagttattaatttgattttagtataatataatgttatgatattttagttaactattatattagatg

SEQ ID NO: 9 - DNA, 24 bp, Artificial Sequence
  Synthetic oligonucleotide
tagtataatataatgctatgatat

SEQ ID NO: 10 - DNA, 22 bp, Artificial Sequence
  Synthetic oligonucleotide
atgttatgacatcttagctaac

SEQ ID NO: 11 - DNA, 54 bp, Artificial Sequence
  Synthetic oligonucleotide
gaacaccatcaacctcgaccaccgcatccgttctcacggaactgctatgtcgat

SEQ ID NO: 12 - DNA, 22 bp, Artificial Sequence
  Synthetic oligonucleotide
accatcaacctcgaacaccgca

SEQ ID NO: 13 - DNA, 21 bp, Artificial Sequence
  Synthetic oligonucleotide
ccatcaacctcgagcaccgca

SEQ ID NO: 14 - DNA, 23 bp, Artificial Sequence
  Synthetic oligonucleotide
caccatcaacctcgatcaccgca

SEQ ID NO: 15 - DNA, 22 bp, Artificial Sequence
  Synthetic oligonucleotide
accatcaacctcgacaaccgca

SEQ ID NO: 16 - DNA, 21 bp, Artificial Sequence
  Synthetic oligonucleotide
ccatcaacctcgacgaccgca

SEQ ID NO: 17 - DNA, 23 bp, Artificial Sequence
  Synthetic oligonucleotide
caccatcaacctcgactaccgca

SEQ ID NO: 18 - DNA, 23 bp, Artificial Sequence
  Synthetic oligonucleotide
caccatcaacctcgaccacagca

SEQ ID NO: 19 - DNA, 22 bp, Artificial Sequence
  Synthetic oligonucleotide
tcgaccaccgcatccgttctca

SEQ ID NO: 20 - DNA, 24 bp, Artificial Sequence
  Synthetic oligonucleotide
acctcgaccaccgcatccgttctc

SEQ ID NO: 21 - DNA, 27 bp, Artificial Sequence
  Synthetic oligonucleotide
acctcgaccaccgcatccgttctcacg

SEQ ID NO: 22 - DNA, 27 bp, Artificial Sequence
  Synthetic oligonucleotide
cgaccaccgcatccgttctcacggaac

SEQ ID NO: 23 - DNA, 22 bp, Artificial Sequence
  Synthetic oligonucleotide
tccgttctcacggaactgctat

SEQ ID NO: 24 - DNA, 22 bp, Artificial Sequence
  Synthetic oligonucleotide
tctcacggaactgctatgtcga

SEQ ID NO: 25 - DNA, 21 bp, Artificial Sequence
  Synthetic oligonucleotide
ttctcacggaactgctatgtc

SEQ ID NO: 26 - DNA, 36 bp, Homosapien
  promoter (0)...(0): DAPK-1 promoter
cggccggggtctgggacggcgacgctcaacggctca

SEQ ID NO: 27 - DNA, 36 bp, Homosapien
  promoter (0)...(0): DAPK-1 promoter
ugguuggggtutgggauggugaugutuaauggutua

SEQ ID NO: 28 - DNA, 36 bp, Homosapien
  promoter (0)...(0): DAPK-1 promoter
taaaccattaaacatcaccatcccaaaccccaacca

SEQ ID NO: 29 - DNA, 36 bp, Homosapien
  promoter (0)...(0): DAPK-1 promoter
cggcuggggtutgggauggcgaugctuaauggctua

SEQ ID NO: 30 - DNA, 36 bp, Homosapien; identical to SEQ ID NO: 28
  promoter (0)...(0): DAPK-1 promoter

SEQ ID NO: 31 - DNA, 22 bp, Artificial Sequence
  Synthetic oligonucleotide
gtaattgtcttaggttgatagg

SEQ ID NO: 32 - DNA, 19 bp, Artificial Sequence
  Synthetic oligonucleotide
tgataggaggagtgtgagg

SEQ ID NO: 33 - DNA, 19 bp, Artificial Sequence
  Synthetic oligonucleotide
gggttgcaattgtcctagg

SEQ ID NO: 34 - DNA, 22 bp, Artificial Sequence
  Synthetic oligonucleotide
ctaaaacaaaaaaaacgcctcc

SEQ ID NO: 35 - DNA, 61 bp, Homosapien
  promoter (0)...(0): DAPK-1 promoter
ggagguguutuuuttgtttuagggguuguaauugaguuagguugauaggaggagtgtgagg

SEQ ID NO: 36 - DNA, 61 bp, Homosapien
  promoter (0)...(0): DAPK-1 promoter
cctcacactcctcctatcaacctaactcaattacaacccctaaaacaaaaaaaacacctcc

SEQ ID NO: 37 - DNA, 61 bp, Homosapien
  promoter (0)...(0): DAPK-1 promoter
ggaggcguutuuuttgtttuagggguugcaauugagcuaggcugauaggaggagtgtgagg

SEQ ID NO: 38 - DNA, 61 bp, Homosapien
  promoter (0)...(0): DAPK-1 promoter
cctcacactcctcctatcagcctagctcaattgcaacccctaaaacaaaaaaaacgcctcc

SEQ ID NO: 39 - DNA, 332 bp, Artificial Sequence
  Internal Control
ctttaaatgctgcattttttatacaatcagaaggtcggttacaaacagccaccggagtagaggtgttgaaattgcaggcaaattgattgtttcaccagctgtattagaagtacaagaaggttggttacaacccaaagacagctgtgcatgaattgcagaaaatttattgcagcttcgccacaggaaaggtggagcaatgtgaagaggcatcatgccatctgctgtaggctatcaaccaatggtaagactcttctggaagcaattgagctatggtcatgccaggtgacaacatatgatgagtcattattatagatggtgtaagtttaggtgca

SEQ ID NO: 40 - DNA, 28 bp, Artificial Sequence
  KERLA-tcdB
ctttaaatgctgcattttttatacaatc

SEQ ID NO: 41 - DNA, 28 bp, Artificial Sequence
  KENP-tcdB
tgcacctaaacttacaccatctataata

SEQ ID NO: 42 - DNA, 24 bp, Artificial Sequence
  NK-toxB-834-AO
gttgttgaattagtatcaactgca

SEQ ID NO: 43 - DNA, 30 bp, Artificial Sequence
  Sign-84-80
gatgcctcttcacattgctccacctttcct

SEQ ID NO: 44 - DNA, 27 bp, Artificial Sequence
  KERLA-Mod1
cattttttatacaatcagaaggtcggt

SEQ ID NO: 45 - DNA, 23 bp, Artificial Sequence
  KERLA-Mod2
tttatacaatcagaaggtcggtt

SEQ ID NO: 46 - DNA, 32 bp, Artificial Sequence
  KERLA-Mod3
cattttttatacaatcagaaggtcggttacaa

SEQ ID NO: 47 - DNA, 257 bp, Homosapien
ctttaaatgctgcattttttatacaatcattaatagaatataatagttctaaagaatctcttagtaatttaagtgtagcaatgaaagtccaagtttacgctcaattatttagtactggtttaaatactattacagatgcagccaaagttgttgaattagtatcaactgcattagatgaaactatagacttacttcctacattatctgaaggattacctataattgcaactattatagatggtgtaagtttaggtgca

(19) United States
(12) Patent Application Publication
(10) Pub. No.: US 2014/0193819 A1
(43) Pub. Date: Jul. 10, 2014

(54) METHODS AND COMPOSITIONS FOR MODULATION OF AMPLIFICATION EFFICIENCY

(71) Applicant: Becton, Dickinson and Company, Franklin Lakes, NJ (US)

(72) Inventors: Tobin Hellyer, Westminster, MD (US); James G. Nadeau, Ellicott City, MD (US)

(73) Assignee: Becton, Dickinson and Company, Franklin Lakes, NJ (US)

(21) Appl. No.: 14/205,229

(22) Filed: Mar. 11, 2014

Related U.S. Application Data
(63) Continuation-in-part of application No. PCT/US2013/067604, filed on Oct. 30, 2013.
(60) Provisional application No. 61/720,959, filed on Oct. 31, 2012.
(60) Provisional application No. 61/779,416, filed on Mar. 13, 2013.

(51) Int. Cl. C12Q 1/68

(52) U.S. Cl. CPC C12Q 1/6858; C12Q 1/6886
USPC 435/6.11; 435/6.12

(57) ABSTRACT

Provided herein are methods and kits for modulating the amplification efficiency of nucleic acids, which are useful in multiplex reactions where the amplification efficiency of one or more nucleic acids in the mixture are desired to be modulated relative to one or more other nucleic acids. Embodiments relate to molecular diagnostics, including detecting sequence variants, such as SNPs, insertions deletions, and altered methylation patterns, as well as the modulation of the amplification efficiency of internal control sequences to provide more accurate control sequences for amplification reactions.

### CROSS-REFERENCE TO RELATED APPLICATIONS

This application claims priority to PCT Application Patent Application No.: PCT/US2013/067604, filed Oct. 30, 2013, which claims priority to U.S. Provisional Patent Application No. 61/720,959, filed Oct. 31, 2012; this application also claims priority to U.S. Provisional Patent Application No. 61/779,416, filed Mar. 13, 2013. The entire contents of each of the preceding applications are hereby incorporated by reference in their entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled GENOM_{—}139A.txt, last saved Mar. 11, 2014, which is 10.2 kb in size. The information is incorporated herein by reference in its entirety.

### BACKGROUND OF THE INVENTION

1. Field of the Invention

The present embodiments relate to methods of modulating the amplification efficiency of nucleic acids. These embodiments are useful in multiplex reactions where the amplification efficiency of one or more nucleic acids in the mixture are desired to be modulated relative to one or more other nucleic acids. Embodiments relate to molecular diagnostics, detecting sequence variants, such as SNPs, insertions deletions, and altered methylation patterns, from samples. Some embodiments disclosed herein can be used to detect (and quantify) sequence variants present in samples that include an excess of wild-type sequences. Some embodiments can be used to modulate the amplification efficiency of control sequences to provide more accurate control sequences for amplification reactions.

2. Description of the Related Art

With the advent of molecular diagnostics and the discovery of numerous nucleic acid biomarkers useful in the diagnosis and treatment of conditions and diseases, detection of nucleic acid sequences, including nucleic acid sequences that may be less abundant or rare in comparison to other sequences in a reaction mixture, has become increasingly important. These less abundant or rare sequences can include sequence variants, mutations and polymorphisms. In many instances, it is desirable to detect sequence variants or mutations (which may in some instances, differ by one a single nucleotide) present in low copy numbers against a high background of wild-type sequences. For example, as more and more somatic mutations are shown to be biomarkers for cancer prognosis and prediction of therapeutic efficacy, the need for efficient and effective methods to detect rare mutations in a sample is becoming more and more important.

In the case in which a target nucleic acid sequence, (e.g., one or more allelic variants) is present in low copy number compared to background sequences (e.g. wild-type), the presence of excess background target sequence creates challenges to the detection of the less abundant target sequence. Nucleic acid amplification/detection reactions almost always are performed using limiting amounts of reagents. A large excess of background target sequences, thus competes for and consumes limiting reagents. As a result amplification and/or detection of less abundant or rare target nucleic acid sequence (e.g. mutant or variant alleles) under these conditions is substantially suppressed, and the methods may not be sensitive enough to detect the less abundant or rare sequences. Various methods to overcome this problem have been attempted. These methods are not ideal, however, because they either require the use of a unique primer for each sequence (e.g., each allele), or the performance of an intricate melt-curve analysis. Both of these shortcomings limit the ability and feasibility of multiplex detection of less abundant or rare sequences (e.g. multiple variant alleles) from a single sample.

A related problem is found in designing appropriate controls to monitor the preanalytical and analytical processes of in vitro diagnostic tests. Such controls frequently involve the use of synthetic nucleotide sequences that are co-amplified with the specific target analyte using the same amplification primers but which are detected by virtue of a difference in the nucleotide sequence in the intervening region between the primers.

In a typical amplification reaction using an internal control sequence, amplification of the native target sequence and the internal amplification control is accomplished with the same set of primers. The intervening region between the amplification primers in the internal amplification control is mutated to permit the hybridization of a specific detector probe that enables the amplification products (amplicons) of the internal amplification control to be distinguished from the amplicons of the native target sequence. A different detector probe is used to detect the presence of amplicons of the native target sequence.

This design scheme often has many drawbacks, most problematic of which is the difference in amplification efficiencies between the native target and the internal amplification control. Design of an appropriate internal amplification control therefore can require significant trial and error.

Some embodiments of the present invention are designed to overcome the many limitations of effecting amplification efficiency that are found in the prior art.

### SUMMARY OF THE INVENTION

Detection of less abundant or rare sequence variants in samples presents numerous challenges. The methods, compositions and kits disclosed herein provide for improved, efficient means to detect less abundant or rare sequences, such as mutations, within a high background of related sequences, such as wild-type allelic sequences, using real-time amplification methods. Also disclosed are methods, compositions and kits for modulating the amplification efficiency of internal control sequences to provide improved internal controls, for example for in vitro diagnostic tests.

One aspect of the embodiments disclosed herein is a method to modulate the amplification efficiency of a nucleic acid sequence in an amplification reaction, the method comprising: providing an amplification reaction comprising a pair of amplification primers comprising a forward primer and a reverse primer, said pair of primers configured to amplify a first target nucleic acid and thereby produce a first amplicon having a first nucleic acid sequence, and configured to amplify a second target nucleic acid and thereby produce a second amplicon having a second nucleic acid sequence, wherein a portion of the second nucleic acid sequence is different from the first nucleic acid sequence; providing a modulator oligonucleotide to the amplification reaction, wherein the modulator oligonucleotide preferentially hybridizes to the second nucleic acid sequence in comparison to the first nucleic acid, and wherein at least a portion of the modulator oligonucleotide shares sequence identity to either the forward or reverse primer, and the remainder of the modulator oligonucleotide hybridizes to at least a portion of the second nucleic acid sequence that differs from the first nucleic acid sequence; amplifying the nucleic acid sequences, wherein the modulator oligonucleotide reduces the amplification efficiency of the second amplicon by competing with at least one of the forward or reverse primer for binding to the second target nucleic acid.

In any of the embodiments disclosed herein, the method may further comprise providing a first reporter probe specific for the first amplicon and a second reporter probe specific for the second amplicon. In any of the embodiments disclosed herein, the first target nucleic acid may be less abundant than the second nucleic acid. In any of the embodiments disclosed herein, the first target nucleic acid may be a rare nucleic acid. In any of the embodiments disclosed herein, the first target nucleic acid may contain an allelic variation and the second target nucleic acid is a wild-type nucleic acid. In any of the embodiments disclosed herein, the second target nucleic acid may be an internal control nucleic acid. In any of the embodiments disclosed herein, the method may further comprise detecting the presence of the first amplicon and/or the second amplicon using a first reporter probe specific for the first amplicon and/or a second reporter probe specific for the second amplicon.

Another aspect of the embodiments disclosed herein relates to a method to detect a first variant target sequence in a sample comprising nucleic acids, the method comprising: providing the sample; contacting the sample with: a pair of amplification primers comprising a forward primer and a reverse primer, said pair of amplification primers configured to amplify a target amplicon, wherein said amplicon comprises a wild-type target sequence or a variant target sequence, and wherein the pair of amplification primers amplifies both wild-type target sequences and variant target sequences; a modulator oligonucleotide that preferentially hybridizes to the wild type target sequence compared to a first variant target sequence under amplification conditions; and a reporter probe, wherein said reporter probe comprises an oligonucleotide that preferentially hybridizes to the first variant target sequence compared to the wild-type target sequence under amplification conditions; wherein said contacting takes place under amplification conditions; and measuring the hybridization of the reporter probe to the first variant target sequence, wherein hybridization of the reporter probe to the first variant target sequence produces a detectable signal indicative of the presence or amount of first variant target species in the sample.

In any of the embodiments disclosed herein, the amplification mixture may comprise extendible molecular species of target amplicons and non-extendible molecular species of target amplicons, wherein a fraction of extendible species (f.e.) represents the fraction of extendible species of a total number target amplicons. In any of the embodiments disclosed herein, the f.e. may be less than about 0.5. In any of the embodiments disclosed herein, the sample may comprise about 100-fold excess of wild-type target sequences compared to variant target sequence. In any of the embodiments disclosed herein, the method may further comprise detecting a second variant target sequence, wherein the modulator oligonucleotide preferentially hybridizes to the wild type target sequence compared to the second variant target sequence under amplification conditions, wherein said method further comprises: contacting the sample with a second reporter probe, wherein said second reporter probe comprises an oligonucleotide that preferentially hybridizes to the second variant target sequence compared to the wild-type target sequence under amplification conditions; wherein said contacting takes place under amplification conditions; and measuring the hybridization of the second reporter probe to the second variant target sequence, wherein hybridization of the reporter probe to the second variant target sequence produces a detectable signal indicative of the presence or amount of second variant target species in the sample. In any of the embodiments disclosed herein, the sample may be simultaneously contacted with the first reporter probe and the second reporter probe. In any of the embodiments disclosed herein, the first and/or second reporter probe may comprise a modified nucleic acid.

In any of the embodiments disclosed herein, the first variant target sequence may be in a gene selected from the group consisting of: KRAS, BRAF, EGFR, TP53, JAK2, NPM1, and PCA3. In any of the embodiments disclosed herein, the second variant target sequence may be in a gene selected from the group consisting of: KRAS, BRAF, EGFR, TP53, JAK2, NPM1, and PCA3. In any of the embodiments disclosed herein, the method may comprise performing real-time PCR. In any of the embodiments disclosed herein, the method may comprise performing isothermal amplification.

Another aspect of the current embodiments is a method for modulating the amplification of a nucleic acid sequence comprising: (a) providing a reaction mixture comprising a sample suspected to contain a target nucleic acid and a primer capable of hybridizing to the target nucleic acid under conditions that will cause at least some of the primer to hybridize to the target nucleic acid if present, wherein the reaction mixture further comprising a modulator oligonucleotide capable of selectively hybridizing to a control nucleic acid, wherein the reaction mixture is subjected to conditions that will cause the modulator oligonucleotide to hybridize to the control nucleic acid; (b) subjecting the reaction mixture to conditions for amplifying the target nucleic acid, if present, and the control nucleic acid, wherein the amplification conditions permit the primer and modulator oligonucleotide to hybridize to the control nucleic acid at similar melting temperatures, wherein the reaction mixture further comprises a first reporter probe specific for the target nucleic acid and a second reporter probe specific for the control nucleic acid; and (c) subjecting the reaction mixture to conditions under which the first reporter probe hybridizes to the target nucleic acid, if present, and the second reporter probe hybridizes to the control nucleic acid wherein the reaction mixture is monitored to detect the hybridization of the respective probes to their respective targets.

Another aspect of the current embodiments is a method of detecting the presence of a methylated cytosine residue in a target DNA sequence in a sample, comprising: treating the sample with a reagent that specifically modifies unmethylated cytosine residues to uracil residues to generate a modified sample DNA to generate a modified sample DNA target sequence; combining the modified sample DNA target sequence with an amplification primer pair comprising a forward primer and a reverse primer, wherein the forward and reverse amplification primers are fully complementary to modified sample DNA that comprises methylated cytosines, and that is not fully complementary to modified sample DNA that comprises uracil residues to create an amplification reaction mixture; contacting the reaction mixture with a reporter probe that is fully complementary to target amplicons generated from modified sample DNA that comprises methylated cytosines, and that is not fully complementary to target amplicons generated from modified sample DNA that comprises uracil; subjecting the reaction mixture to an amplification reaction to generate target amplicons; detecting the amount of reporter probe bound to target amplicons produced from the amplification reaction.

In any of the embodiments disclosed herein, the reaction mixture may further comprise a modulator oligonucleotide that competes with the reverse primer and/or the reporter probe for hybridizing to the amplified target sequence, wherein the modulator oligonucleotide preferentially hybridizes to amplicons produced form modified sample DNA that comprises uracil residues. In any of the embodiments disclosed herein, the modulator oligonucleotide may be between 15 and 30 nucleotides in length. In any of the embodiments disclosed herein, the first and/or second reporter probe may be between 15 and 30 nucleotides in length. In any of the embodiments disclosed herein, the modulator oligonucleotide may be longer than the first and/or second reporter probe. In any of the embodiments disclosed herein, the first and/or second reporter probe may not be overlapping with either the forward or reverse amplification primer. In any of the embodiments disclosed herein, the first and/or second reporter probe may be overlapping with the modulator oligonucleotide, wherein the overlap between the first and/or second reporter probe and the modulator oligonucleotide does not extend to the 3′ end of the reporter probe. In any of the embodiments disclosed herein, the first and/or second reporter probe may be overlapping with the modulator oligonucleotide, wherein the overlap between the first and/or second reporter probe and the modulator oligonucleotide does not extend to the 5′ end of the modulator oligonucleotide. In any of the embodiments disclosed herein, the overlap between the first reporter and/or second probe and the modulator oligonucleotide may not extend to the 5′ end of the modulator oligonucleotide. In any of the embodiments disclosed herein, the modulator oligonucleotide may be overlapping with either the forward or reverse amplification primer, and wherein the overlap does not extend to the 3′ end of the modulator oligonucleotide. In any of the embodiments disclosed herein, the overlap between the modulator oligonucleotide and the forward or reverse amplification primer may not extend to the 5′ end of the forward or reverse amplification primer. In any of the embodiments disclosed herein, the first reporter probe and/or second may be selected from the group consisting of a TAQMAN® reporter probe, a SCORPION® reporter probe, a hybridization (FRET) probe, and a molecular beacon probe.

In another aspect, embodiments disclosed herein relate to a method for modulating the amplification of a nucleic acid control sequence. Part of the method include providing a reaction mixture comprising a sample suspected to contain a target nucleic acid and primer capable of hybridizing to the target nucleic acid. Hybridization may occur under conditions that will cause at least some of the primer to hybridize to the target nucleic acid if present, wherein the reaction mixture also contains a modulator oligonucleotide capable of selectively hybridizing to a control nucleic acid. The reaction mixture is subjected to conditions that may cause the modulator oligonucleotide to hybridize to the control nucleic acid.

### BRIEF DESCRIPTION OF THE DRAWINGS

FIG. 1 is a schematic of an exemplary embodiment illustrating a method using modulator oligonucleotides to control the amplification efficiency in a multiplex amplification reaction.

FIG. 2 is a schematic of an exemplary embodiment illustrating a method for detection of a single, rare variant allele according to the embodiments disclosed herein.

FIG. 3 is a schematic of an exemplary embodiment illustrating a method for the simultaneous detection of more than one rare, variant allele according to the embodiments disclosed herein.

FIGS. 4A-C are a schematic of an exemplary embodiment illustrating a method for the detection of methylation variants according to the embodiments disclosed herein.

FIGS. 5A-D is a schematic showing the different, possible species of molecular complexes in in reaction mixtures containing an analyte (A), amplification primer (P), modulator/blocker oligonucleotide (B), detector probe (D) and polymerase (E).

FIG. 6 shows the equilibrium between the various species of molecular complexes shown in FIGS. 5A-D.

FIG. 7 illustrates an exemplary method to estimate the fraction extendible target species, “f.e.,” of the complexes shown in FIG. 5, according to the embodiments disclosed herein.

FIG. 8 shows a mathematical model for an amplification reaction on a sample comprising two different target species, according to the embodiments disclosed herein.

FIG. 9 is a schematic of an exemplary embodiment illustrating a design of an internal control that is amplified using the same primers as the native target sequence.

FIG. 10 is a schematic of an exemplary embodiment illustrating a method using modulator oligonucleotides to suppress amplification of an internal control sequence in a multiplex reaction.

FIG. 11 depicts the various reporter probes, modulator/blocker oligonucleotides and forward amplification primers used in the simulated real-time PCR assays discussed in EXAMPLE 1.

FIGS. 12A-B show simulated amplification curves of real-time amplification reactions using the various conditions described in EXAMPLE 1, with a mixture of wild-type and G34T mutant KRAS nucleic acids, present in a ratio of 10000:100 (wt:mutant). FIG. 12A shows the amplification curve (relative fluorescence v. cycle number) of the reaction under the described parameters, wherein the W.T. f.e., as explained in EXAMPLE 1, is approximately 0.159. FIG. 12B shows the amplification curve (relative fluorescence v. cycle number) of the reaction under the described, wherein the WT f.e. is approximately 0.717, as described in EXAMPLE 1.

FIG. 13A depicts a target region of the DAPK-1 promoter region as described in EXAMPLE 2, including the location of cytosine residues that are potentially methylated. CpG sites are boxed.

FIG. 13B depicts a schematic showing a reaction to detect methylation variants in the DAPK-1 promoter, as described in EXAMPLE 2.

FIG. 14 is a schematic of an exemplary embodiment illustrating a method using modulator oligonucleotides to suppress amplification of an internal control sequence in an assay for the C. difficile toxin B gene.

FIG. 15 depicts exemplary embodiments of modulator oligonucleotides employed in the present technology.

### DETAILED DESCRIPTION OF THE PREFERRED EMBODIMENT

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Modulation of Amplification Efficiency

The embodiments disclosed herein relate to methods, compositions, uses and kits for modulating the amplification efficiency of one or more nucleic acid sequences. Preferably, the modulation occurs in a multiplex reaction where a pair of amplification primers is used to amplify more than one target nucleic acid sequence, such as a wild-type sequence and mutant or variant alleles, or a target sequence and an internal control sequence. Modulation of the amplification efficiency is accomplished using one or more “modulator oligonucleotides” also referred to as a “blocking oligonucleotides” or “blocker oligonucleotides” as explained in more detail herein. In one aspect, embodiments disclosed herein provide improved methods for detection of mutant or variant alleles. Some embodiments disclosed herein advantageously overcome many of the limitations of previous methods of molecular detection of rare mutations, and enable detection of multiple alleles within a single real-time PCR reaction, without the requirement for multiple, allele-specific amplification primers.

Modulator oligonucleotides disclosed herein can be used with any type of nucleic acid amplification reaction in which primer extension occurs. Examples of such reactions, include, but are not limited to, Polymerase Chain Reaction (PCR), Strand Displacement Amplification (SDA), Transcription-Mediated Amplification (TMA), Nucleic Acid Sequence Based Amplification (NASBA), Loop Mediated Amplification (LAMP), Smart Amplification Process (SMAP), Ligase Chain Reaction (LCR), and Helicase Dependent Amplification (HAD).

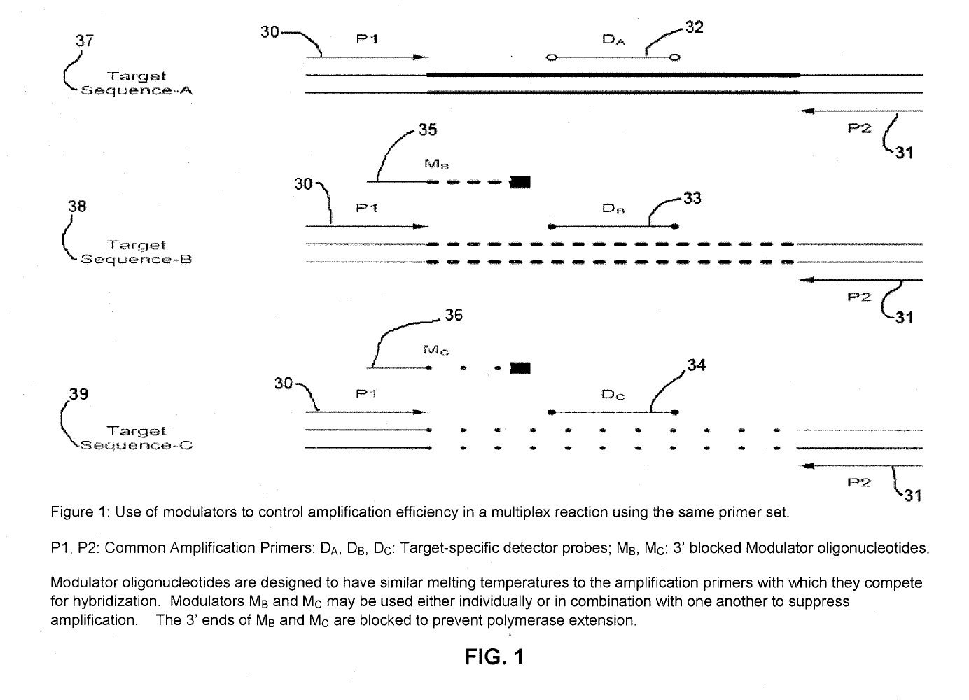

A general embodiment of a modulator oligonucleotide is described in FIG. 1. Modulator oligonucleotides can be designed to regulate the amplification efficiency of selected nucleic acid sequences in any multiplexed reaction that employs one or more common amplification primers. As long as there is adequate sequence heterogeneity between several target sequences in one reaction mixture, amplification efficiency of one or more of the target sequences can be affected/controlled by modulator oligonucleotides. For example with reference to FIG. 1, if three target sequences, A 37, B 38, and C 39, are amplified using common amplification primers P1 30 and P2 31, the amplification efficiency of one or more of the target sequences can be affected by modulator oligonucleotides, e.g., M_{B} 35 for Target Sequence-B 38 or M_{C} 36 for Target Sequence-C 39. The modulator oligonucleotides M_{B} 35 and M_{c} 36 both contain nucleotide sequences that hybridize to a region of their respective targets that overlaps the target binding region of the amplification primer P1 30 and the intervening region in the respective target sequences (e.g., Target Sequence-B 38 or Target Sequence-C 39). The intervening region is the nucleotide sequence between the target binding region for amplification primer P1 30 and the target binding region for amplification primer P2 31. Amplification efficiency is monitored by target-specific detector probes for each target sequence, D_{A} 32, D_{B} 33, and D_{C} 34.

As explained in more detail herein, the modulator oligonucleotide is modified such that it cannot support primer extension by the polymerase in the reaction. Because the modulator oligonucleotides 35 and 36 compete with the hybridization of the amplification primer 30, and do not support polymerase extension, the amplification efficiency of Target Sequence-B 38, and Target Sequence-C 39, are reduced relative to their efficiency in the absence of the modulator oligonucleotides 35 and 36. In this way, the amplification efficiencies of the three Target Sequences A, B, and C can be adjusted relative to each other.

This modulation of the relative amplification efficiency of one or more target nucleic acid sequences in a multiplex reaction has numerous uses. For example, where a particular target sequence in an amplification reaction is less abundant or rare, it will often be difficult to detect its presence in the reaction because the more abundant sequence will be amplified to such an extent that the less abundant or rare sequence will not be detectable. By modifying the relative amplification efficiency of the sequences in the reaction, for example by using a modulator oligonucleotide that is specific for the more abundant sequence, it is possible to detect the less abundant or rare sequence in the amplification reaction. By way of illustration, in FIG. 1, Target Sequence-A 37, could be a rare sequence, while Target Sequences-B 38 and -C 39, are the abundant sequences.

One skilled in the art is aware of the many factors that affect the efficiency with which two oligonucleotides will anneal to each other or to a particular target. One important factor, but not the only one, is the temperature at which two sequences will anneal. For example, to obtain similar reaction kinetics for the modulator oligonucleotides and the primer under the conditions of amplification, the melting temperature (Tₘ) of the modulator oligonucleotide could be similar to that of the primer with which it is designed to compete. Furthermore, the primer and modulator concentrations should also be similar. Methods for calculating primer Tₘ are described by von Ahsen et al., Clin. Chem. 11: 1956-1961 (2011).

In addition to Tₘ, another condition which may affect the efficiency with which two oligonucleotides anneal to each other or to a particular target is the relative concentrations of the particular oligonucleotides. For example, a higher concentration of modulator oligonucleotide compared to primer, may yield an increased hybridization of the modulator to target. The same may be true for the reverse scenario. Design of modulator oligonucleotides and calculation of concentrations for use in reactions are described in more detail herein.

### Detection of Variant or Mutant Alleles and Methylation Patterns

In some embodiments, modulator oligonucleotides are utilized to analyze a sample for less abundant or rare sequences in the presence more abundant sequences. In one embodiment, the less abundant or rare sequence is one or more allelic variants within a target sequence. Allelic variants have been implicated in genetic disorders, susceptibility to different diseases, responses to various therapeutics and the like. Accordingly, the importance of detection of allelic variants or mutations in target sequences cannot be underestimated. The term “target sequence” generally refers to a nucleic acid sequence of interest, e.g., a genomic DNA, an mRNA, a cDNA, or the like, to be queried for the presence of allelic variants, e.g., rare allelic variants or mutations. As used herein, the term “rare sequence,” or “rare allelic variant” or “variant target sequence,” refers to a target sequence that is present at a lower copy number in a sample compared to an alternative sequences, particularly alternative allelic variant, such as a wild-type target sequence. For example, the rare target sequence may be present in a sample at a frequency of less than 1/10, 1/100, 1/1,000, 1/10,000, 1/100,000, 1/1,000,000, 1/10,000,000, 1/100,000,000, 1/1,000,000,000, or less (or any frequency in between), compared to another allelic variant or wild-type target sequence. For example, a rare sequence, e.g. a rare allelic variant or variant target sequence, may be present at less than 2, 3, 4, 5, 6, 7, 8, 9, 10, 20, 30, 40, 50, 60, 70, 80, 90, 100, 200, 300, 500, 750, 1000, 2500, 5000, 7500, 10000, 25000, 50000, 75000, 100000, 250000, 500000, 750000, 1000000, or more, copies in a sample. In some embodiments, the term allelic variant can refer to single nucleotide polymorphisms, substitutions, insertions, deletions, or the like.

The compositions and methods disclosed herein can be used in the detection of numerous allelic variants, including nonsense mutations, missense mutations, insertions, deletions, and the like. Owing to the advantageous sensitivity and specificity of detection afforded by the compositions and methods disclosed herein, the presence of a rare sequence such as an allelic variant within a sample can be detected, amongst a high wild-type background. Accordingly, although the skilled artisan will appreciate that the methods disclosed herein can be used in a variety of settings to detect, e.g., germline mutations, the methods are particularly well-suited for use in the detection of somatic mutations, such as mutations present in tumors. Non-limiting examples of rare, somatic mutations useful in the diagnosis, prognosis, and treatment of various tumors include, for example, mutations in ABL, AKT1, AKT2, ALK, APC, ATM, BRAF, CBL, CDH1, CDKN2A, CEBPA, CRLF2, CSF1R, CTNNB1, EGFR, ERBB2, EZH2,FBXW7, FGFR, FGFR2, FGFR3, FLT3, FOXL2, GATA1, GATA2, GNAQ, GNAS, HNF1A, HRAS, IDH1, IDH3, JAK2, KIT, KRAS, MEK1, MET, MPL, NF2, NOTCH1, NOTCH2, NPM, NRAS, PCA3, PDGFRA, PIK3CA, PIK3R1, PIK3R5, PTCH1, PTEN, PTPN11, RB1, RET, RUNX1, SMAD4, SMARCB, SMO, STK11, TET2, P53, TSHR, VHL, WT1, and others. Exemplary mutant alleles associated with cancer useful in the embodiments disclosed herein include, but are not limited to those described in publications listed on the world wide web site for COSMIC (Catalogue Of Somatic Mutations In Cancer) available at sanger.ac.uk/genetics/CGP/cosmic/add info.

DNA methylation is an important mechanism of epigenetic gene regulation. Rare changes in the DNA methylation patterns of genes associated with cell growth and differentiation have been linked to a variety of cancers. As such, detection of rare, altered DNA methylation patterns offers potential in cancer diagnosis, treatment and therapeutic monitoring. By way of example, epigenetic silencing of tumor suppressor genes through hypermethylation of their promoter regions is frequently associated with the onset of disease and detection of such changes may have utility in early diagnosis. Accordingly, in some embodiments, the methods and compositions disclosed herein can be advantageously used to detect rare, altered DNA methylation patterns, e.g., to enhance the specificity of detection of low levels of DNA methylation in a background of high levels of unmethylated DNA, to enhance the sensitivity and specificity of detection of rare methylation events, and/or to enhance the detection of unmethylated DNA or loss of methylation in a background of highly methylated DNA. Non-limiting examples of variations in DNA methylation that can be advantageously queried using the methods described herein include, but are not limited to the detection of methylation of the promoter region of Human Death Associated Kinase Protein-1 (DAKP-1) gene, promoter in genes involved in cell cycle, growth differentiation and development (e.g., BRCA1, CCNA, CCND2, CDKN1C, CDKN2A (p14ARF), CDKN2A (p16), SFN, TP73, and the like), cell adhesion genes, e.g., CDH1, CDH13, OPCML (aOBCAM), PCDH10 and the like; transcription factors, e.g., ESR1, HIC1, PRDM2, RASSF1, TP73, HIC1, HNF1B, RUNX3, WT1.; hormone receptors, e.g., ESR1; drug metabolism genes, e.g., GSTP1, and the like; genes involved in apoptosis and anti-apoptosis, e.g., PYCARD, TNFRSF10C, TNFRSF10D, APC and the like, phosphatases, e.g., PTEN, DNA methylation, e.g., MGMT, PRDM2; extracellular matrix molecules, e.g., ADAM23, SLIT2, THBS1, as well as other genes, e.g., RASSF1, and the like; miRNAs, e.g., let-7g, mir-10a, mir-124-2, mir-126, mir-149, mir-155, mir-15b Cluster (mir-15b, mir-16-2), mir-17 cluster (mir-17, mir-18a, mir-19a, mir-19b-1, mir-20a, mir-92a-1), miR-191 Cluster (miR-191, miR-425), mir-210, mir-218-1, mir-218-2, mir-23b Cluster (mir-23b, mir-24-1, mir-27b), mir-301a, mir-30c-1 Cluster (mir-30c-1, mir-30e), mir-32, mir-378, mir-7-1, and the like.

The methods and compositions disclosed herein can be used to analyze nucleic acids of samples. The term “sample” as described herein can include bodily fluids (including, but not limited to, blood, urine, feces, serum, lymph, saliva, anal and vaginal secretions, perspiration, peritoneal fluid, pleural fluid, effusions, ascites, and purulent secretions, lavage fluids, drained fluids, brush cytology specimens, biopsy tissue (e.g., tumor samples), explanted medical devices, infected catheters, pus, biofilms and semen) of virtually any organism, with mammalian samples, particularly human samples.

In some embodiments, the sample is processed prior to the nucleic acid testing. For example, in some embodiments, the sample is processed to extract and/or separate and/or isolate nucleic acids from other material present in the sample. In some embodiments, the sample is analyzed directly, e.g., without prior nucleic acid extraction and/or isolation. In some embodiments, the sample is processed in order to isolate genomic DNA. In some embodiments, the sample is processed in order to isolate mRNA. In some embodiments, the sample is processed by using RT-PCR to generate cDNA, prior to the nucleic acid testing. Methods for processing samples and nucleic acids in accordance with the methods disclosed herein are well-known, and are described, e.g., in Current Protocols in Molecular Biology, Greene Publ. Assoc. Inc. & John Wiley & Sons, Inc., NY, N.Y.; Sambrook et al. (1989) Molecular Cloning, Second Ed., Cold Spring Harbor Laboratory, Plainview, N.Y.); Maniatis et al. (1982) Molecular Cloning, Cold Spring Harbor Laboratory, Plainview, N.Y.; and elsewhere.

Detection of Sequence Variants

Provided herein are methods and compositions useful in the detection of sequence variants, e.g. insertions, deletions, nonsense mutations, missense mutations, and the like. In the methods for detecting allelic variants or variant target sequences disclosed herein, the sample, which comprises the nucleic acids to be analyzed, are contacted with an amplification primer pair, comprising a forward primer and a reverse primer that flank the target sequence or target region containing a sequence of interest (e.g., a wild-type, mutant, or variant allele sequence) to be analyzed, also referred to herein as the “intervening sequence.” By “flanking” the target sequence, it is understood that the variant or wild-type allelic sequence is located between the forward and reverse primers, and that the binding site of neither the forward nor reverse primer comprises the rare sequence, variant allelic sequence or wild-type allelic sequence to be assessed. For example, in some embodiments, the variant or wild-type allelic sequence to be assessed is removed from or positioned away from the 3′ end of either oligonucleotide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, e.g., 100 or more, 200 or more, 300 or more, 400 or more, 500 or more, etc., nucleotides. Amplification primers that flank, but that do not overlap with, the rare target sequence, variant target sequence, or the wild-type target sequence are thus not “allele-specific” amplification primers, and are capable of amplification of various different alleles or variants of a sequence of interest. Thus, in some embodiments, the amplification primers are configured to amplify various mutant or variant alleles and wild type alleles non-preferentially. As discussed in further detail herein, the addition of modulator oligonucleotides to an amplification reaction suppresses the amplification of one or more of the target sequences, typically the wild-type target sequences, and enables preferential amplification of a less abundant or rare sequence, typically the non-wild-type, e.g., variant, mutant or rare variant alleles.

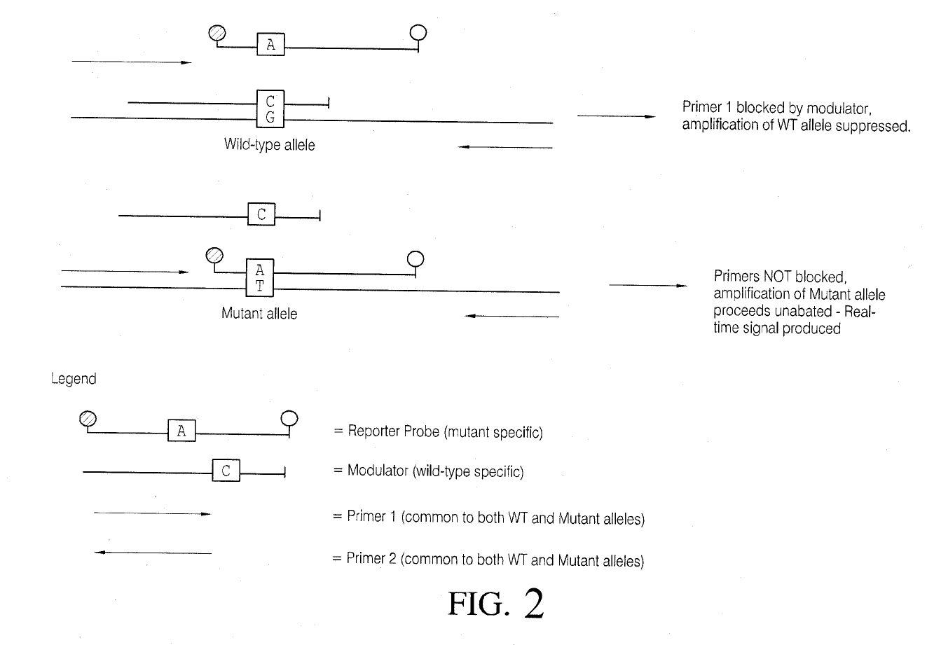

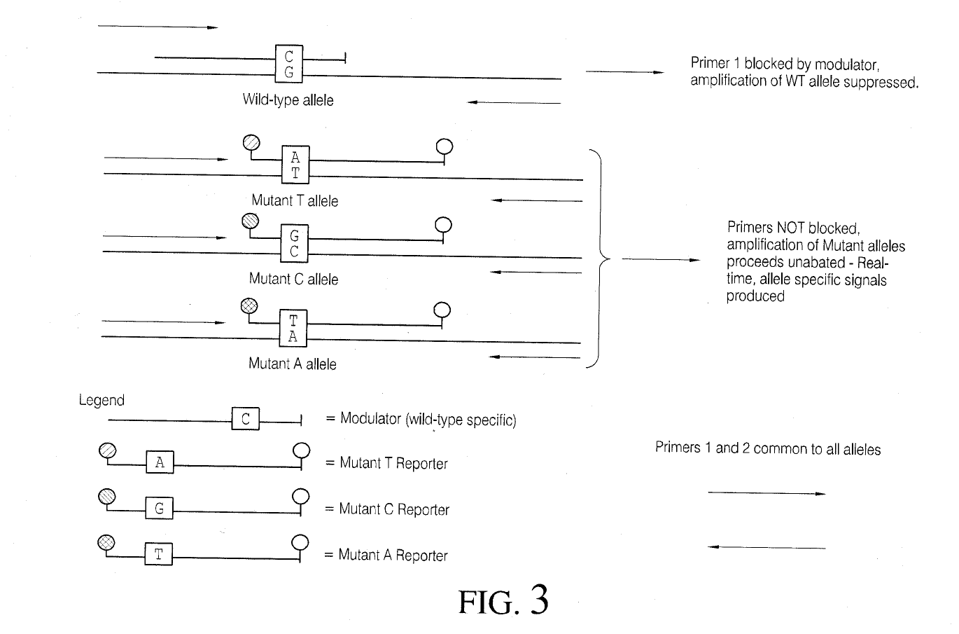

FIGS. 2 and 3 are depictions of exemplary methods according to the embodiments disclosed herein for the detection of sequence variants. As shown in FIGS. 2 and 3, amplification primers (forward primer 1 and reverse primer 2) flank the wild type and mutant allele sequences of interest, and comprise sequences common to both wild-type and mutant or variant allele sequences. Accordingly, as shown in FIG. 3, in contrast to methods that utilize allele-specific amplification primers to achieve preferential amplification of rare sequences, the present methods advantageously enable the simultaneous amplification of multiple variant sequences, using a single amplification primer pair. One of skill in the art will recognize that the embodiments disclosed in FIGS. 2 and 3 are generally applicable to detection of multiple sequences in a reaction, and are not limited to detection of mutant alleles.

Detection of Altered DNA Methylation Patterns

Also provided herein are embodiments related to methods and compositions for the detection of DNA methylation variants (DNA that has an altered methylation pattern), e.g., is methylated at cytosine residues that are non-methylated in wild-type DNA, or includes unmethylated cytosine residues that are methylated in wild-type DNA.

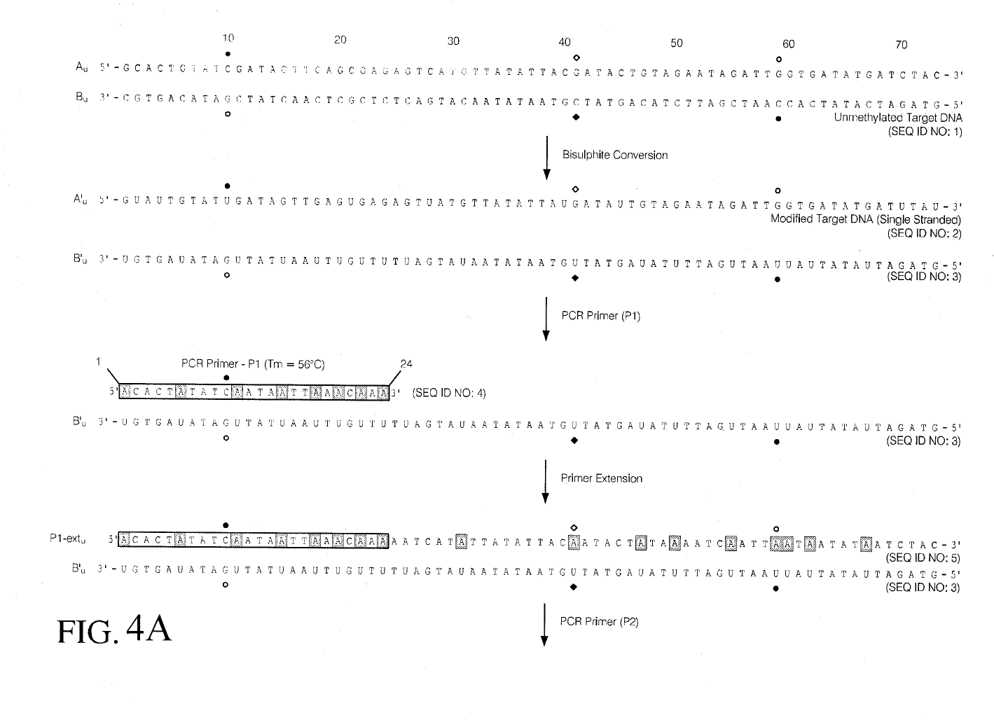

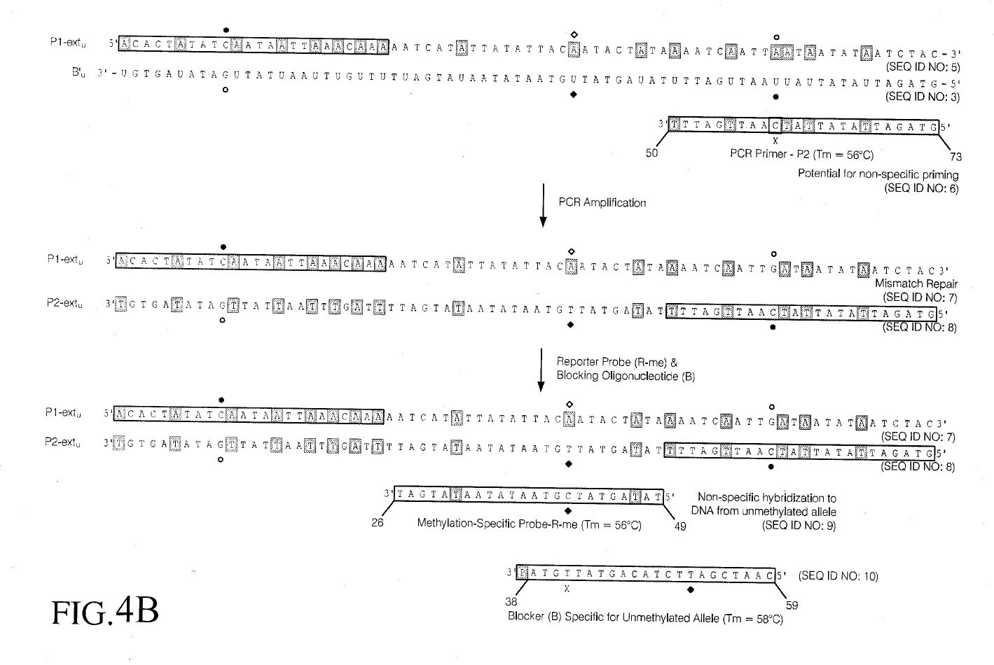

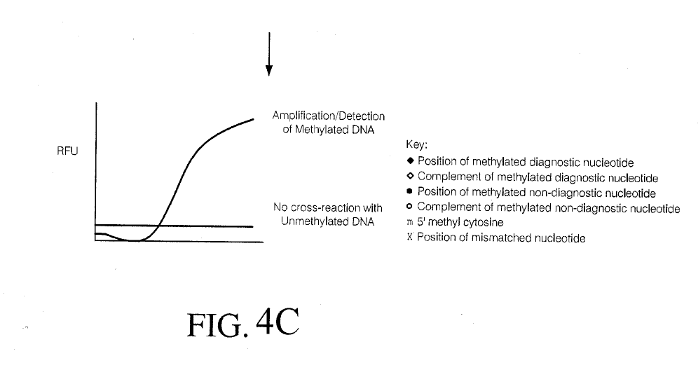

In some embodiments, the sample DNA is treated with an agent the selectively modifies unmethylated cytosine residues. By way of example only, in some embodiments, the sample nucleic acids are treated with sodium bisulphite, according to art-accepted methods. (See, e.g., Formmer, et al. (1992) Proc. Nat. Acad. Sci. USA 89:1827-1831). Treatment with sodium bisulphite sulphonates unmethylated cytosines, but not methylated cytosines. Following sulphonation, the sample is subjected to conditions (e.g., alkaline conditions, or any other appropriate conditions), that deaminate the sulphonated DNA to yield a uracil-bisulphite derivative that is in turn converted to uracil by alkaline desulphonation. Selective conversion of the unmethylated cytosine residues on both strains (the first strand and the second strand) generates novel sequences, referred to as “modified target DNA,” for convenience, as illustrated in FIGS. 4A-C. The modified sample nucleic acids are then subjected to an amplification (and/or detection) reaction, as discussed below.

In some embodiments, provided herein are methods to detect, or enhance the specificity of detection of rare methylation events, e.g., by performing a methylation-specific amplification reaction (e.g., methylation specific PCR). Modified sample nucleic acids are contacted with a forward and a reverse amplification primer that specifically hybridize to opposite strands of the modified sample nucleic acids—the forward primer hybridizes to the first strand of the modified nucleic acids (e.g., modified sample nucleic acids, or modified target DNA) and the reverse primer hybridizes to the second strand of the modified nucleic acids (e.g., modified sample nucleic acids, or modified target DNA)—and amplify the region between the two primers under amplification conditions.

Referring to FIGS. 4A-C, the forward primer (P1), comprises a sequence that is complementary to and specifically hybridizes to modified target DNA B, the target nucleotide sequence of the second strand following cytosine modification—the unique sequence generated by specific modification of unmethylated cytosine residues as discussed above. The forward primer thus contains one or more adenine residues that are located in the primer to hybridize to uracil residues present in the modified sample nucleic acids (e.g., modified sample nucleic acids, or modified target DNA). Accordingly, in some embodiments, the forward primer comprises one or more adenine residues that will base-pair with uracil residues in the second strand template sequence, modified target DNA B (converted from unmethylated cytosine residues in the second strand original sample sequence). In some embodiments, the one or more adenine residues that base-pair with uracil residues in the template sequence include an adenine residue located at the 3′ end of the forward primer P1, as shown in FIGS. 4-C. As such, extension will occur when the original sample DNA prior to modification of the unmethylated cytosines (e.g., by bisulphite treatment), comprises an unmethylated cytosine residue at the same position (shown in FIG. 4A-C). If the second strand of the template contains methylated cytosine residues, then treatment with bisulphite will not generate a novel sequence, and the adenosine residues in the methylation-specific primer will be mismatched with the methylated cytosines in the second strand of the template nucleic acids. As such, amplification will not occur when the second strand of the original sample nucleic acids (prior to modification) comprises a methylated cytosine residue at the same position (not shown). In some embodiments, the forward primer is fully complementary to a target sequence that comprises methylated cytosines and is also fully complementary to a target sequence that comprises unmethylated cytosines (see, e.g., EXAMPLE 2, below). For example, in some embodiments, the forward primer hybridizes to a target sequence that does not include potentially methylated cytosine residues.

In some embodiments, the reverse primer (depicted as P2 in FIGS. 4A-C) is complementary to the unique first strand sequence generated by amplification from the forward primer following modification of the sample nucleic acids. The unique first strand sequence generated by amplification is depicted as P1-extᵤ in FIGS. 4A-C. Accordingly, in some embodiments, the reverse primer comprises one or more thymine residues, which correspond to the position of one or more uracil residues (converted from unmethylated cytosine residues in the second strand original sample sequence, modified target DNA B), and that base-pair with adenine residues present in the extension product from the forward primer (P1-extᵤ). In some embodiments, the one or more thymine residues corresponding to the position of one or more uracil residues (converted from unmethylated cytosine residues in the second strand original sample sequence), is at the 3′ end of the reverse primer. As such, extension will occur when the second strand of the original sample DNA comprises an unmethylated cytosine residue at the same position (shown in FIGS. 4-C), and will not occur when the second strand of the original sample DNA comprises a methylated cytosine residue at the same position (not shown). The extension product from P2 is depicted as P2-extᵤ in FIGS. 4A-C.

In some embodiments, the methods comprise contacting the treated sample (e.g., a sample that has been treated to selectively modify cytosine residues) with methylation-specific forward and reverse primers as described herein, under amplification conditions, as described below. In some embodiments, the methods include contacting the treated sample with a methylation-specific probe (e.g., by including the methylation-specific probe in the reaction mixture prior to amplification, or by contacting the sample with the methylation-specific probe post-amplification). Methylation-specific probes can include sequences that are complementary to and thus hybridize to the unique amplicons produced by successful extension from the forward and reverse methylation-specific primers, as described above. In some embodiments, the methylation specific probe comprises one or more cytosine residues that correspond to the position of a methylated cytosine residue present in the sample nucleic acids (e.g., and that are thus present as cytosine residues on the P2-extᵤ strand, or second strand of the amplified, modified target sequences). As shown in FIGS. 4A-C, the methylated cytosine residues are not converted to uracil by bisulphite treatment, and thus the first and second strands of the amplicons produced by P1 and P2 (P1-extᵤ and P2-extᵤ, respectively, in FIGS. 4A-C) contain a guanine-cytosine base pair. In some embodiments, the methylation specific probe (shown as Rₘₑ in FIGS. 4A-C) also contains one or more thymine residues that correspond to the position of an unmethylated cytosine residue in the sample nucleic acids (and thus, a uracil residue in the modified sample nucleic acids, modified target DNA B). In some embodiments, the methylation-specific probe contains a detectable label or detectable moiety, as discussed in further detail below.

In some embodiments, the amplification reaction mixture also includes a “modulator oligonucleotide,” also referred to as a “blocking oligonucleotide” or “blocker oligonucleotide.” In some embodiments, modulator oligonucleotides are used selectively suppress non-specific hybridization of the methylation-specific amplification primers and/or methylation-specific reporter probes. Accordingly, modulator oligonucleotides can be used to overcome the potential for false positive results owing to the presence of mixed populations of methylated and unmethylated target nucleic acid sequences, as may be encountered in clinical samples. As shown in FIG. 4, in some embodiments, a blocker oligonucleotide is used to enhance the specificity of methylation-specific amplification. For example, in some embodiments, the blocker oligonucleotide (shown as “B” in FIG. 4B) competes with both primer P2 and/or the reporter probe Rₘₑ for hybridization with the amplified target. The sequence of the modulator oligonucleotide or blocker oligonucleotide B is designed such that it preferentially hybridizes, in this case, to amplification product derived from unmethylated DNA target strand Aᵤ. The Tₘ of the modulator/blocker oligonucleotide B is designed to be substantially similar to the Tₘ of the forward and/or reverse methylation-specific amplification primers, and/or reporter probe (P1 and P2, and, reporter probe Rₘₑ). In some embodiments, the Tₘ of the blocker oligonucleotide differs by less than 15° C., 14° C., 13° C., 12° C., 11° C., 10° C., 9° C., 8° C., 7° C., 6° C., 5° C., 4° C., 3° C., 2° C., or 1° C., or less, from the methylation-specific amplification primers and/or reporter probe. As such, in some embodiments, the reactions are optimized to allow discrimination between methylated an unmethylated DNA forms, e.g., by balancing concentration and the conditions of hybridization (in particular temperature and salt concentration, as well as other factors known in the art). In general, the higher the Tₘ of the blocker oligonucleotide relative to that of the primer and/or reporter probe with which it competes, the lower the concentration of blocker oligonucleotide required to suppress non-specific amplification and/or detection of target nucleic acids. As discussed in further detail herein, the modulator/blocker oligonucleotides are designed such that they cannot be extended from their 3′ ends.

### Amplification Primers

Amplification primers useful in the embodiments disclosed herein are preferably between 10 and 45 nucleotides in length. For example, the primers can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers can be provided in any suitable form, included bound to a solid support, liquid, and lyophilized, for example. In some embodiments, the primers and/or probes include oligonucleotides that hybridize to a reference nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “standard PCR conditions” include, for example, any of the PCR conditions disclosed herein, or known in the art, as described in, for example, PCR 1: A Practical Approach, M. J. McPherson, P. Quirke, and G. R. Taylor, Ed., (c) 2001, Oxford University Press, Oxford, England, and PCR Protocols: Current Methods and Applications, B. White, Ed., (c) 1993, Humana Press, Totowa, N.J. The amplification primers can be substantially complementary to their annealing region, comprising the specific variant target sequence(s) or the wild type target sequence(s). Accordingly, substantially complementary sequences can refer to sequences ranging in percent identity from 100, 99, 98, 97, 96, 95, 94, 93, 92, 91, 90, 89, 85, 80, 75 or less, or any number in between, compared to the reference sequence. Conditions for enhancing the stringency of amplification reactions and suitable in the embodiments disclosed herein, are well-known to those in the art. A discussion of PCR conditions, and stringency of PCR, can be found, for example in Roux, K. “Optimization and Troubleshooting in PCR,” in PCR PRIMER: A LABORATORY MANUAL, Diffenbach, Ed. © 1995, Cold Spring Harbor Laboratory Press, Cold Spring Harbor, N.Y.; and Datta, et al. (2003) Nucl. Acids Res. 31(19):5590-5597.

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50 □C; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42□C; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42□C, with washes at 42□C in 0.2×SSC (sodium chloride/sodium citrate) and 50% formamide at 55□C, followed by a high-stringency wash consisting of 0.1×SSC containing EDTA at 55□C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37□C in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50□C. The skilled artisan will recognize how to adjust the temperature, ionic strength, etc. as necessary to accommodate factors such as oligonucleotide length and the like.

In some embodiments, primer pairs comprising a forward and reverse primer are used in the amplification methods described herein, e.g., to produce target amplicons. In some embodiments, the Tₘ of the forward and reverse primers are substantially similar, e.g., differ by less than 15° C., 14° C., 13° C., 12° C., 11° C., 10° C., 9° C., 8° C., 7° C., 6° C., 5° C., 4° C., 3° C., 2° C., or 1° C., or less.

### Modulator Oligonucleotides

In an amplification reaction wherein reagents such as polymerase and dNTPs are limiting, when a sample comprises a large excess of wild-type target sequences compared to less abundant or rare sequences, such as variant or mutant target sequences or alleles, (e.g., 10 fold, 100 fold, 1000 fold or more excess of wild-type target sequence compared to variant or mutant sequence), the kinetics of the amplification reaction are driven such that the limiting reagents are consumed in the amplification of the more abundant, e.g. wild-type sequences, while amplification and/or detection of the less abundant or rare sequence, e.g., rare variant, rare mutant, alleles. is suppressed. As discussed herein, in order to shift the equilibrium to favor amplification of the rare sequence, e.g. variant or mutant alleles, modulator oligonucleotides, also referred to as blocker oligonucleotides, can be added to the reaction.

As used herein, the term “modulator oligonucleotide” (also referred to as “blocker oligonucleotide”) refers to an oligonucleotide that binds to a strand of DNA within the target amplicon, and that is designed to preferentially bind to the target sequence whose amplification is to be reduced. Typically, this is the more abundant sequence, such as the wild-type allele sequence (e.g., the abundant allelic sequence, such as a wild-type allele sequence) compared to the target variant sequence (e.g., the rare allelic variant). The modulator oligonucleotide generally comprises a modification, or modifications, as discussed herein, that prevent primer extension by a polymerase. Thus, a modulator oligonucleotide can tightly bind to a particular sequence, typically the wild type allele, in order to suppress amplification of that sequence, while amplification of the less abundant or rare, e.g. variant target allele sequence, is allowed to occur. As explained herein, modulator oligonucleotides can also be advantageously used in the methods described herein for the detection of methylation variants, e.g., in methylation specific amplification reactions as discussed above. Similarly, as disclosed herein, modulator oligonucleotides can also be used in conjunction with internal control sequences to provide improved internal controls.

Modulator oligonucleotides as disclosed herein refer to oligonucleotides that are incapable of extension by a polymerase, for example, when hybridized to its complementary sequence in an amplification assay, e.g., PCR. Several different means of modifying oligonucleotides to render them incapable of extension by a polymerase are known and useful in the embodiments disclosed herein. By way of example, common examples of oligonucleotide modifications include, for example, 3′-OH modifications and dideoxy nucleotides. Numerous 3′-OH blocking materials are known and suitable, and include cordycepin (3′-deoxyadenosine) and other 3′-moieties such as those described in Josefen, M. et al. (2009) Mol. Cell. Probes 23:201-223 McKinzie, P. et al. (2006) Mutagenesis, 21(6):391-397; Parson, B. et al. (2005) Methods Mol. Biol., 291:235-245; Parsons, B. et al. (1992) Nucl. Acids. Res., 25:20(10):2493-2496, and Morlan, J. et al. (2009) PLoS One 4(2):e4584, the disclosures of which relating to oligonucleotide modifications are hereby incorporated by reference. In some embodiments, the 3′-OH is blocked with a (3-amino-2-hydroxy)-propoxyphosphoryl. In some embodiments, the 3′-OH is blocked by introduction of a 3′-3′-A-5′ linkage such as those described in U.S. Pat. No. 5,660,989. Incorporating a 3′ phosphate, inverted base (linked 3′-5′), 3′ biotin, or addition of 3′ tail of non-complementary bases (e.g., oligo-dT) to the modulator oligonucleotide can also block polymerase extension.

In some embodiments, the modulator oligonucleotide comprises a moiety that binds within the minor groove of double-stranded DNA at its 3′ end, which prevents polymerase extension. A variety of moieties that bind to the minor groove of DNA suitable for the modulator oligonucleotides disclosed herein are known in the art, and include, but are not limited to those described in U.S. Pat. No. 5,801,155, Wemmer, et al. (1997) Curr. Opin. Structural Biol. 7:355-361, Walker, et al. (1997) Biopolymers 44:323-334, Zimmer, et al. (1986) Molec. Biol. 47:31-112, and Reddy, B. et al. (1999) Pharmacol. Therap. 84:1-111. Methods for incorporating or attaching minor-groove binding moieties to oligonucleotides are well-known. For example, methods described in U.S. Pat. Nos. 5,512,677, 5,419,966, 5,696,251, 5,585,481, 5,492,610, 5,736,626, 5,801,155 and 6,727,356 are suitable for modifying oligonucleotides to generate a modulator/blocker oligonucleotide.

In some embodiments, the modulator oligonucleotides disclosed herein can include a minor-groove binding moiety located at the 5′ end, the 3′ end, or at a position within the oligonucleotide.

The skilled artisan will readily appreciate that the exemplary “blocking” modifications discussed above are provided by way of illustration only, and that any blocking modification known or discovered in the future can be used in the modulator/blocker oligonucleotides and methods disclosed herein.

In some embodiments, the modulator oligonucleotides comprise one or more modifications that increase the Tₘ of the oligonucleotide. For example, in some embodiments the modulator oligonucleotide can comprise one or more nucleosidic bases different from the naturally occurring bases (adenine, cytosine, thymine, guanine and uracil). In some embodiments, the modified bases effectively hybridize to nucleic acid units that contain naturally occurring bases. In some embodiments, the modified base(s) increase the difference in the Tₘ between matched and mismatched sequences, and/or decrease mismatched priming efficiency, thereby improving the specificity and sensitivity of the assay.

Non-limiting examples of modified bases useful in the embodiments disclosed herein include the general class of base analogues 7-deazapurines and their derivatives and pyrazolopyrimidines and their derivatives (described in PCT WO 90/14353; and U.S. application Ser. No. 09/054,630, the disclosures of each of which are incorporated herein by reference in regards to the base analogues). Examples of base analogues of this type include, for example, the guanine analogue 6-amino-1H-pyrazolo[3,4-d]pyrimidin-4(5H)-one (ppG), the adenine analogue 4-amino-1H-pyrazolo[3,4-d]pyrimidine (ppA), and the xanthine analogue 1H-pyrazolo[4,4-d]pyrimidin-4(5H)-6(7H)-dione (ppX). These base analogues, when present in an oligonucleotide of some embodiments of the methods and compositions disclosed herein, strengthen hybridization.

Additionally, in some embodiments, modified sugars or sugar analogues can be present in one or more of the nucleotide subunits of a modulator oligonucleotide. Sugar modifications useful in the embodiments disclosed herein include, but are not limited to, attachment of substituents to the 2′, 3′ and/or 4′ carbon atom of the sugar, different epimeric forms of the sugar, differences in the α or β-configuration of the glycosidic bond, and other anomeric changes. Sugar moieties useful in the embodiments disclosed herein include, but are not limited to, pentose, deoxypentose, hexose, deoxyhexose, ribose, deoxyribose, glucose, arabinose, pentofuranose, xylose, lyxose, and cyclopentyl.

In some embodiments the modulator oligonucleotide can contain one or more locked nucleic acid (LNA)-type modifications. LNA modifications useful in the embodiments disclosed herein can involve alterations to the pentose sugar of ribo- and deoxyribonucleotides that constrains, or “locks,” the sugar in the N-type conformation seen in A-form DNA. In some embodiments, this lock can be achieved via a 2′-O, 4′-C methylene linkage in 1,2:5,6-di-O-isopropylene-.alpha.-D-allofuranose. In other embodiments, this alteration then serves as the foundation for synthesizing locked nucleotide phosphoramidite monomers. (See, for example, Wengel J., Ace. Chem. Res., 32:301-310 (1998), U.S. Pat. No. 7,060,809; Obika, et al., Tetrahedron Lett 39: 5401-5405 (1998); Singh, et al., Chem Commun 4:455-456 (1998); Koshkin, et al., Tetrahedron 54: 3607-3630 (1998), the disclosures of each of which are incorporated herein by reference

In some embodiments, modified bases useful in the embodiments disclosed herein include 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-O,4′-C-ethylene bridged nucleic acid (ENA) bases. Other examples of modified bases that can be used in the embodiments disclosed herein are described in U.S. Pat. No. 7,517,978 (the disclosure of which is incorporated herein by reference).

Many modified bases, including for example, LNA, ppA, ppG, 5-Fluoro-dU (fdU), are commercially available and can be used in oligonucleotide synthesis methods well known in the art. In some embodiments, synthesis of modified primers and probes can be carried out using standard chemical means also well known in the art. For example, in certain embodiments, the modified moiety or base can be introduced by use of a (a) modified nucleoside as a DNA synthesis support, (b) modified nucleoside as a phosphoramidite, (c) reagent during DNA synthesis (e.g., benzylamine treatment of a convertible amidite when incorporated into a DNA sequence), or (d) by post-synthetic modification according to art-accepted techniques.

In some embodiments, the modulator oligonucleotides are synthesized so that the modified bases are positioned at the 3′ end of the modulator oligonucleotide. In some embodiments, the modified base are located between, 1-6 nucleotides, e.g., 2, 3, 4 or 5 nucleotides away from the 3′-end of the modulator oligonucleotide.

Modified internucleotide linkages can also be present in oligonucleotides, e.g., the modulator oligonucleotides in the embodiments disclosed herein. Modified linkages useful in the embodiments disclosed herein include, but are not limited to, peptide, phosphate, phosphodiester, phosphodiester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art.

In some embodiments, the modulator oligonucleotide binds to a sequence which overlaps with the annealing region of the forward or reverse amplification primer. For example, in some embodiments, the modulator oligonucleotide and the forward or reverse primer are identical across 5, 6, 7, 8, 9, 10, 11, 12, 13, 14, 15, 16, 17, 18, 19, 20, 21, 22, 23, 24, 25, or more consecutive nucleotides. In some embodiments, the overlap in sequence identity between the modulator oligonucleotide and the forward or reverse amplification primer exists over 10%, 15%, 20%, 25%, 30%, 35%, 40%, 45%, 50%, 55%, 60%, 65%, 70%, 75%, 80%, 85%, or more, or any percentage in between, of the length of the modulator oligonucleotide and/or amplification primer. In some embodiments, the amplification primer comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 5′ end that are not identical to the modulator oligonucleotide (but that are complementary or substantially complementary to the target intervening sequence). In some embodiments, the modulator oligonucleotide comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 3′ end that are not identical to the amplification primer (but that are complementary or substantially complementary to the target intervening sequence).

As shown in FIGS. 2 and 3, the modulator oligonucleotide preferentially binds to the more abundant, e.g. wild-type target sequence compared to the rare, e.g. mutant or variant target sequence. Also shown in FIGS. 2 and 3 is the overlap between the amplification primer (primer 1 as shown) and the modulator oligonucleotide. As shown in FIGS. 2 and 3, binding of the modulator oligonucleotide to the wild type allele target sequence prevents binding and extension of the amplification primer, thereby suppressing amplification of the wild-type sequence. In contrast to the wild-type allele sequence, the amplification primer will preferentially bind to the mutant allele sequence, over the modulator oligonucleotide. Thus, the amplification is not blocked and the amplification of the mutant target allele sequence proceeds unimpeded. By this means, the present method and compositions advantageously allows for simultaneous and preferential amplification of one or more sequences relative to another. In one embodiment, the present method and compositions advantageously allows for simultaneous and preferential amplification of less abundant or rare variant or mutant target allele sequences.

### Reporter Probes

To detect the presence and/or amount of multiple target sequences in the sample, for example variant target sequence(s), including rare variant or mutant template nucleic acids, the sample is contacted with one or more sequence-specific reporter probes, also referred to herein as detector probes, e.g. allele-specific probes. In some embodiments, the methods disclosed herein provide for the detection of more than one variant or mutant allele sequence in a sample. Accordingly, in some embodiments, a sample can be contacted with 1, 2, 3, 4, 5, 6, 7, 8 or more, reporter probes. Each reporter probe preferentially binds to a cognate allelic variant compared to the wild type allelic sequence. As discussed herein, in some embodiments, reporter probes can be advantageously used to detect methylation variants, e.g., in methylation-specific amplification as discussed above.

The reporter/detector probes can comprise a detectable moiety. In some embodiments, the probe can include a detectable label. Labels of interest include directly detectable and indirectly detectable radioactive or non-radioactive labels such as fluorescent dyes and the like. Directly detectable labels refer to detectable moieties that provide a directly detectable signal without interaction with one or more additional chemical agents. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase.

In some embodiments, the label is a directly detectable label. Directly detectable labels of particular interest include fluorescent labels. Fluorescent labels suitable in the detector probes of the embodiments disclosed herein include fluorophore moieties. Specific fluorescent dyes of interest include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3′,3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R),5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like. In preferred embodiments, the reporter/detector probe can be a molecular beacon probe, a TAQMAN™ probe, or a SCORPION™ probe.

In some embodiments, the reporter probe(s) have a Tₘ that is higher than the Tₘ of the forward and reverse amplification primers used in the methods disclosed herein. For example, in some embodiments, the probes, e.g., molecular beacon probes or the like, have a Tₘ that is greater than 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C., or more than either amplification primer used to generate an amplicon to which the oligonucleotide probe hybridizes. For example, a molecular beacon probe can have a Tₘ that is at least 5-10° C. higher than either amplification primer pair used to generate the amplicon to which the molecular beacon hybridizes. In some embodiments, the reporter probe(s) have a Tₘ that is the same or lower than the forward and reverse amplification primers disclosed herein.

As used herein, the term “Tm” and “melting temperature” are interchangeable terms which refer to the temperature at which 50% of a population of double stranded polynucleotide molecules become dissociated into single strands. The Tm of particular nucleic acids, e.g., primers, or oligonucleotide probes, or the like can be readily calculated by the following equation: Tm=69.3+0.41×(G+C) %−650/L, wherein L refers to the length of the nucleic acid. The Tm of a hybrid polynucleotide may also be estimated using a formula adopted from hybridization assays in 1 M salt, and is commonly used for calculating the Tm for PCR primers: [(number of A+T)×2° C.+(number of G+C)×4° C.], see, for example, Newton et al. (1997) PCR (2nd ed; Springer-Verlag, New York). Other more sophisticated computations exist in the art, which take structural as well as sequence characteristics into account for the calculation of Tm. A calculated Tm is merely an estimate; the optimum temperature is commonly determined empirically.

In some embodiments, the reporter probe can comprise an oligonucleotide that is shorter in length than the forward or reverse amplification primer. For example, in some embodiments, the reporter probe(s) is 1, 2, 3, 4, 5, 6, 7, 8, 9, 10, or more nucleotides shorter than either the forward or reverse amplification primer.

In some embodiments, the reporter probe(s) hybridize to a sequence that overlaps with at least a portion of hybridization site of the modulator oligonucleotide. For example, in some embodiments, the reporter probe(s) and the modulator oligonucleotide are identical across 5, 6, 7, 8, 9, 10, 11, 12, 13, 14, 15, 16, 17, 18, 19, 20, 21, 22, 23, 24, 25, or more consecutive nucleotides. In some embodiments, the overlap in sequence identity between the reporter probe(s) and the modulator oligonucleotide exists over 10%, 15%, 20%, 25%, 30%, 35%, 40%, 45%, 50%, 55%, 60%, 65%, 70%, 75%, 80%, 85%, or more, or any percentage in between, of the length of the modulator oligonucleotide and/or reporter probe(s). In some embodiments, the modulator oligonucleotide comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 5′ end that are not identical to the reporter probe (but that are complementary or substantially complementary to the target sequence). In some embodiments, the reporter probe comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 3′ end that are not identical to the modulator probe (but that are complementary or substantially complementary to the target sequence).

As shown in FIGS. 2 and 3, the reporter probe(s) is preferably allele-specific. That is, the reporter probe is complementary to the variant or mutant allele sequence(s) being assayed, and non-complementary to the wild-type allele sequence. As shown in FIGS. 2 and 3, binding of the detector probe to the mutant or variant target allele sequence preferably does not block or impede amplification by the amplification primers. Binding of the reporter probe to the mutant allele sequence (e.g., within sample template sequence or amplicon sequences) produces a detectable signal. As shown in FIG. 3, in some embodiments, reaction mixtures can contain more than one detector probe, wherein each detector probe is specific for a different variant or mutant target allele sequence, and wherein each detector probe comprises a different detectable moiety. Accordingly, detection and identification of different mutant alleles in a single sample/reaction mixture is possible. One of skill in the art will recognize that the embodiments disclosed in FIGS. 2 and 3 are generally applicable to detection of multiple sequences in a reaction, and are not limited to mutant alleles.

In addition to the sample, amplification primers, modulator oligonucleotide, and reporter probe(s), the reaction mixture includes a polymerase. The skilled artisan will appreciate that many polymerases known to those in the art are suitable for the methods described herein. For example, thermostable polymerases (including commercially available polymerases) obtained from Thermus aquaticus, Thermus thermophilus, Thermococcus litoralis, Pyrococcus furiosus, Pyrococcus woosii and other species of the Pyrococcus genus, Bacillus stearothermophilus, Sulfolobus acidocaldarius, Thermoplasma acidophilum, Thermus flavus, Thermus ruber, Thermus brockianus, Thermotoga neapolitana, Thermotoga maritima and other species of the Thermotoga genus, and Methanobacterium thermoautotrophicum, and mutants of each of these species are useful in the embodiments disclosed herein. Preferable thermostable polymerases can include, but are not limited to, Taq DNA polymerase, Th DNA polymerase, Tma DNA polymerase, or mutants, derivatives or fragments thereof.

Usually the reaction mixture will further comprise four different types of dNTPs corresponding to the four naturally occurring nucleoside bases: dATP, dTTP, dCTP, and dGTP. In the disclosed methods, each dNTP will typically be present in an amount ranging from about 10 to 5000 μM, usually from about 20 to 1000 μM, about 100 to 800 μM, or about 300 to 600 μM.

The reaction mixture can further include an aqueous buffer medium that includes a source of monovalent ions, a source of divalent cations, and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc, and the like, where the cation will typically be magnesium. Any convenient source of magnesium cation may be employed, including magnesium chloride, magnesium acetate, and the like. The amount of magnesium present in the buffer may range from 0.5 to 10 mM, and can range from about 1 to about 6 mM, or about 3 to about 5 mM. Representative buffering agents or salts that may be present in the buffer include Tris, Tricine, HEPES, MOPS, and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, for example, about pH 6.0, 6.5, 7.0, 7.5, 8.0, 8.5, 9.0, or 9.5. Other agents that may be present in the buffer medium include chelating agents, such as EDTA, EGTA, and the like. In some embodiments, the reaction mixture can include BSA, or the like. In addition, in some embodiments, the reactions can include a cryoprotectant, such as trehalose, particularly when the reagents are provided as a master mix, which can be stored over time.

In preparing a reaction mixture, the various constituent components may be combined in any convenient order. For example, the buffer may be combined with primer, polymerase, and then template nucleic acid, or all of the various constituent components may be combined at the same time to produce the reaction mixture.

Alternatively, commercially available premixed reagents can be utilized in the methods disclosed herein, according to the manufacturer's instructions, or modified to improve reaction conditions (e.g., modification of buffer concentration, cation concentration, or dNTP concentration, as necessary), including, for example, TAQMAN® Universal PCR Master Mix (Applied Biosystems), OMNIMIX® or SMARTMIX® (Cepheid), IQ&#8482; Supermix (Bio-Rad Laboratories), LIGHTCYCLER® FastStart (Roche Applied Science, Indianapolis, Ind.), or BRILLIANT® QPCR Master Mix (Stratagene, La Jolla, Calif.).

The reaction mixture can then be subjected to amplification, or primer extension conditions. For example, in some embodiments, the reaction mixture is subjected to thermal cycling or isothermal amplification. Thermal cycling conditions can vary in time as well as in temperature for each of the different steps, depending on the thermal cycler used as well as other variables that could modify the amplification's performance. In some embodiments, a 2-step protocol is performed, in which the protocol combines the annealing and elongation steps at a common temperature, optimal for both the annealing of the primers and probes as well as for the extension step. In some embodiments, a 3-step protocol is performed, in which a denaturation step, an annealing step, and an elongation step are performed.

In some embodiments, the compositions disclosed herein can be used in connection with devices for real-time amplification reactions, e.g., the BD MAX® (Becton Dickinson and Co., Franklin Lakes, N.J.), the VIPER® (Becton Dickinson and Co., Franklin Lakes, N.J.), the VIPER LT® (Becton Dickinson and Co., Franklin Lakes, N.J.), the SMARTCYLCER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (BioRad Laboratories, Hercules, Calif.), IMX4000® (Stratagene, La Jolla, Calif.), CFX96™ Real-Time PCR System (Bio-Rad Laboratories Inc.), and the like.

In some embodiments, the compositions disclosed herein can be used in methods comprising isothermal amplification of nucleic acids. Isothermal amplification conditions can vary in time as well as temperature, depending on variables such as the method, enzyme, template, and primer or primers used. Examples of amplification methods that can be performed under isothermal conditions include, but are not limited to, some versions of LAMP, SDA, and the like.

Isothermal amplification can include an optional denaturation step, followed by an isothermal incubation in which nucleic acid is amplified. In some embodiments, an isothermal incubation is performed without an initial denaturing step. In some embodiments, the isothermal incubation is performed at least about 25° C., for example about 25° C., 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75° C., including ranges between any of the listed values. In some embodiments, the isothermal incubation is performed at about 37° C. In some embodiments, the isothermal incubation is performed at about 64° C. In some embodiments, the isothermal incubation is performed for 180 minutes or less, for example about 180, 165, 150, 135, 120, 105, 90, 75, 60, 45, 30, or 15 minutes, including ranges between any two of the listed values.

In some embodiments, the accumulation amplicons of the target sequences, e.g. the variant or mutant target allele sequence(s) are monitored in real-time. Methods for monitoring and assaying amplification reactions in real-time are widely known, and the skilled artisan will appreciate that any of the art-accepted techniques of real-time amplification are suitable for use in the embodiments disclosed herein. Exemplary descriptions of real-time amplification useful in the embodiments disclosed herein can be found, for example, in U.S. Pat. No. 6,783,984; U.S. Pat. No. 6,303,305, and the like. As used herein, the term “Ct” or “Ct value” refers to threshold cycle and signifies the cycle (or fractional cycle) of an amplification assay in which signal from a reporter that is indicative of amplicon generation (e.g., fluorescence), first become detectable above a background level. In some embodiments, the threshold cycle or “Ct” is the cycle number at which nucleic acid amplification becomes exponential. In some embodiments, e.g., in embodiments wherein amplification proceeds via isothermal amplification, threshold time values are used to signify the time in an amplification assay in which signal from a reporter that is indicative of amplicon generation (e.g., fluorescence), first becomes detectable above a background level. In some embodiments, the threshold time value is the time at which nucleic acid amplification becomes exponential.

As used herein, the term “delta Ct” or “ΔCt” refers to the difference in the numerical cycle number at which the signal passes a fixed threshold between two different samples or reactions. In some embodiments ΔCt refers to the difference in numerical cycle number at which exponential amplification is reached between two different samples or reactions. The ΔCt can be used to identify the specificity between a matched reporter probe to the corresponding target nucleic acid sequence and a mismatched reporter probe to the same corresponding sequence.

Various methods to calculate Ct values and threshold time values are known in the art and are useful in the embodiments disclosed herein. By way of example only, methods described in U.S. Pat. Nos. 6,783,984, 6,303,305, and the like can be used in calculating Ct values and threshold time values in the methods disclosed herein. Accordingly, in some embodiments, the methods include the step of determining the Ct value or threshold time value, for each target allele sequence of interest (e.g., mutant or target allele sequences).

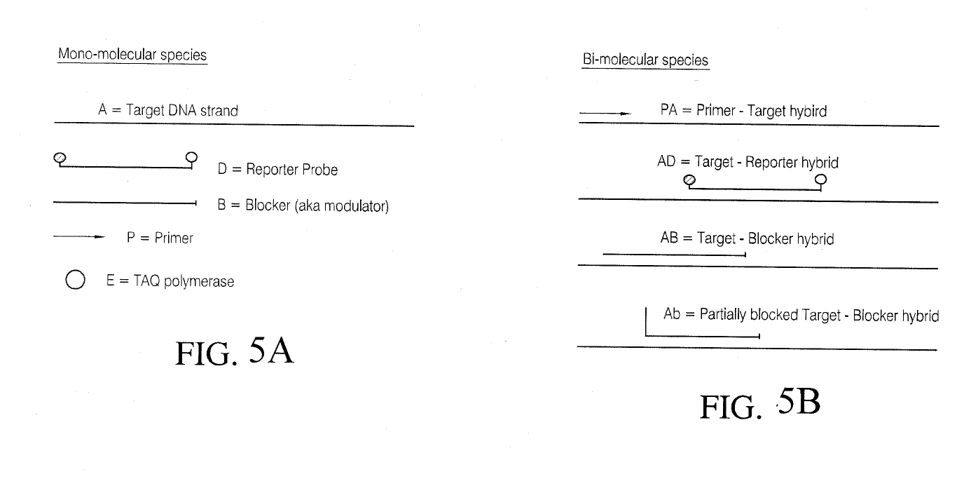

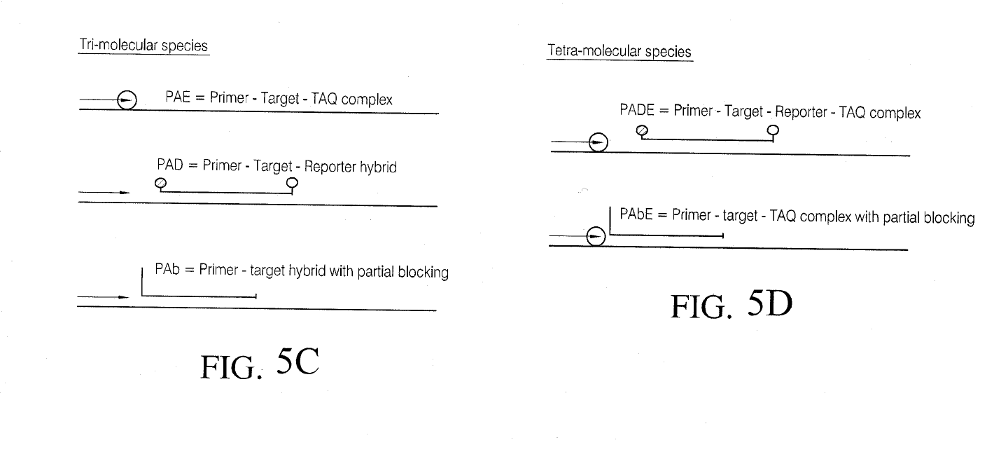

The present embodiments are based, in part, upon the recognition that using a combination of amplification primers, oligonucleotide modulators, and allele-specific detector probes, one can render amplification of less abundant or rare sequences, e.g. rare alleles, thermodynamically more favorable, thereby enabling their detection in samples that contain predominantly a background sequence, e.g. wild-type or other variant allele sequences. FIGS. 5-8 illustrate the concepts described herein, including the thermodynamic consideration used in practicing the embodiments disclosed herein.

FIG. 5 depicts the molecular species present in a reaction mixture that is subjected to primer extension or amplification conditions. “A” represents the “analyte” or target region of interest that comprises either the wild-type or variant or mutant allele sequence. As shown in FIG. 5A, the molecular species in the reaction mixture include the analyte, the reporter probe (“D”), the modulator/blocker oligonucleotide (“B”), the amplification primer(s) (“P”), and the polymerase (“E”). FIG. 5B shows bi-molecular species, including amplification primer bound to its cognate sequence on the analyte (“PA”), reporter probe bound to its cognate sequence on the analyte (“DA”), modulator/blocker oligonucleotide bound to its cognate sequence on the analyte (wild-type target allele sequence) (“BA”), and modulator/blocker oligonucleotide that is partially bound to the analyte (variant or mutant target allele sequence) (“Ab”). FIG. 5C depicts tri-molecular species, such as (1) complexes between the amplification primer, its cognate analyte, and polymerase (“PAE”); (2) complexes between the amplification primer, its cognate analyte, and a reporter probe (“PAD”); and (3) complexes between the amplification primer, its cognate analyte and an oligonucleotide modulator/blocker (“PAb”). FIG. 5D depicts possible tetra-molecular species, including (1) complexes between an amplification primer, its cognate analyte sequence, reporter probe, and polymerase (“PADE”); and (2) complexes between an amplification primer, its cognate analyte, a modulator/blocker oligonucleotide, and polymerase (“PAbE”). The PAb and PAbE species represent the case in which nucleotide at and near the 5′ end of the modulator/blocker are unhybridized to the analyte, but the remaining nucleotides of the modulator/blocker are hybridized to the analyte. In all cases, primers, probes, modulators/blockers may hybridize with wild-type or variant DNA; however the perfectly matched hybrids (e.g. modulator/blocker with wild-type DNA) will be thermodynamically more stable than hybrids containing mismatches (e.g. modulator/blocker with variant DNA).

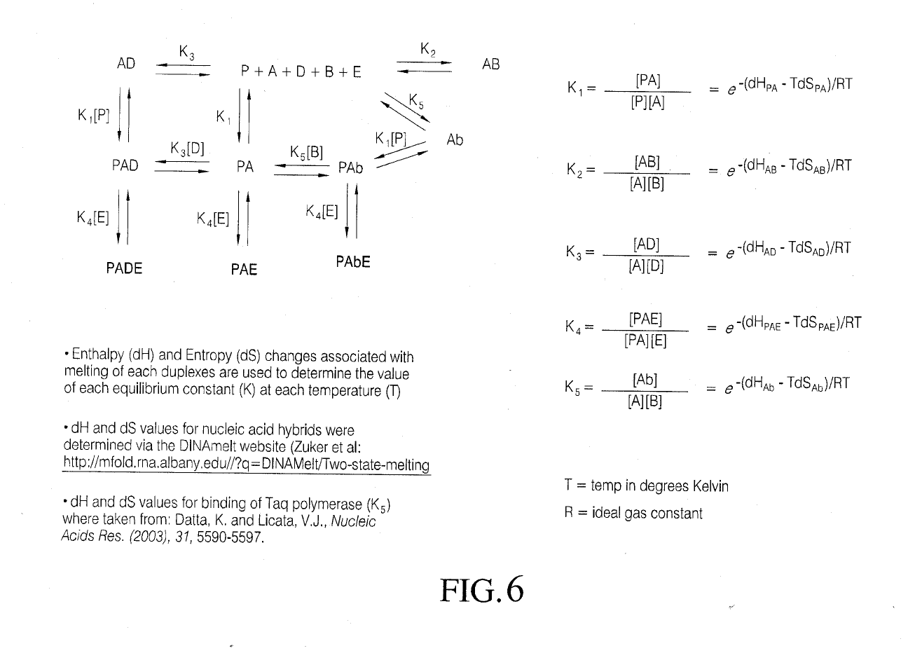

The molecular complexes shown in FIGS. 5A-4D exist in a multi-state equilibrium, as shown in FIG. 6. The association between each of the mono-molecular species is described by an equilibrium constant, K. The embodiments disclosed herein area based, in part, upon the discovery that equilibrium constants for the various molecular species shown in FIG. 5 can be advantageously used to model reaction conditions to maximize amplification of rare sequences, e.g. rare variant or mutant allele sequences, compared in samples comprising an excess of copies (e.g., 5×, 10×, 20×, 30×, 40×, 50×, 100×, 500×, 750×, 1000×, or greater) of more abundant sequences, e.g. wild-type allele sequence, compared to a less abundant or rare sequence, while minimizing detrimental effects on amplification efficiency. In accordance with the methods disclosed herein, the equilibrium constants for the complexes depicted in FIG. 5 can be estimated using enthalpy (dH) and entropy (dS) changes associated with melting of each of the duplexes, at each temperature. dH and dS values for each hybrid can be estimated or calculated using any art-accepted methods. By way of example, dH and dS can be calculated using publicly available algorithms, such as those available on the world wide web site hypertext transfer protocol://mfold.rna.albany.edu//?q=DINAMelt/Two-state-melting. The skilled artisan will appreciate that many known algorithms for calculation of dH and dS can be used in the methods disclosed herein. FIG. 6 shows the calculation of individual equilibrium constants according to the methods disclosed herein.

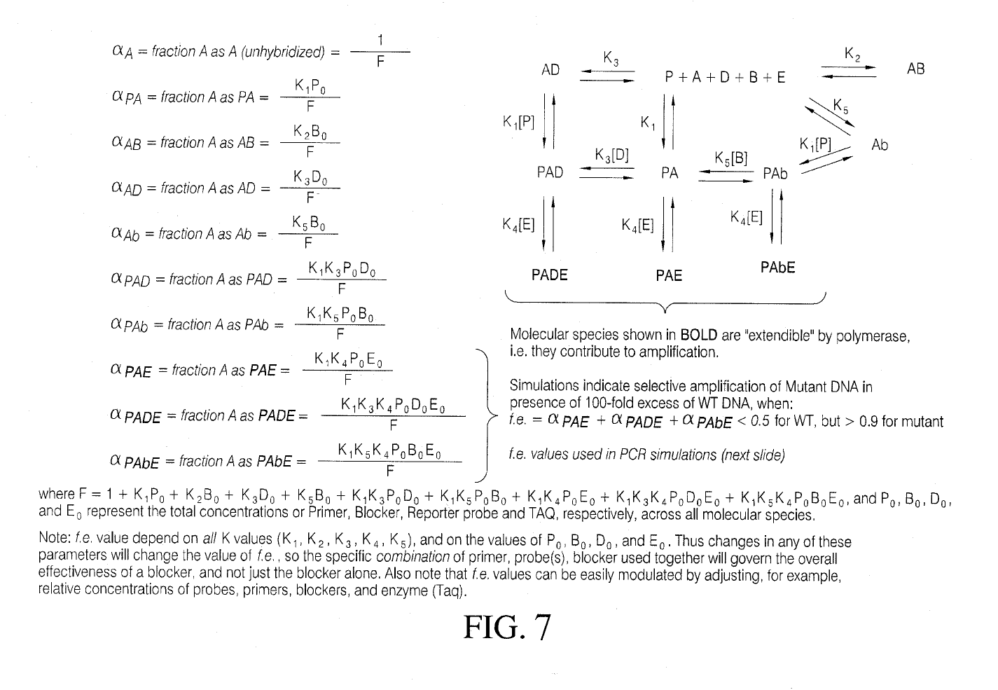

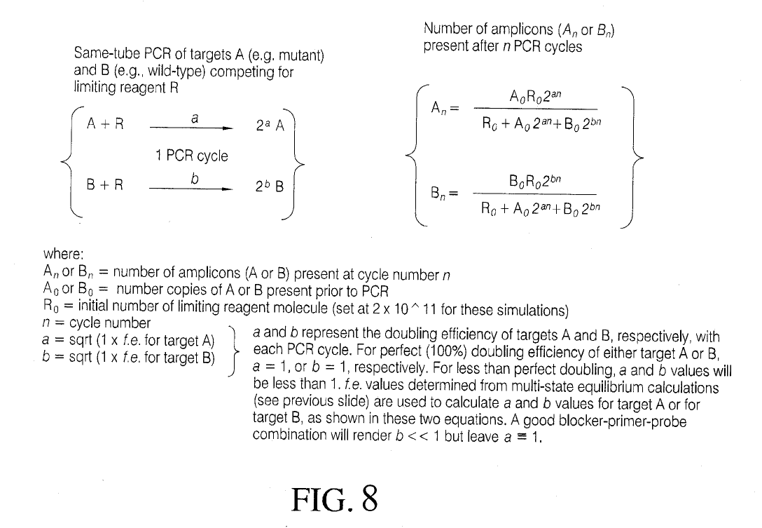

Equilibrium constants can be used to estimate the fraction of analyte “A” bound to modulator oligonucleotide, detector probes, and amplification primers, in a reaction mixture, e.g., in multi-state equilibrium, and that these values are useful in methods of maximizing amplification of rare allele sequences. The fraction of analyte, represented by “a” in various complexes within the reaction can be determined using the equations shown in FIG. 7, using the starting concentrations of amplification primer (P₀), modulator/blocker oligonucleotide (B₀), detector probe (D₀), and polymerase (E₀), and the respective equilibrium constants, K₁-K₅, for each of the different complexes, as discussed in connection with FIG. 6. FIG. 8 shows a model estimator for the number of amplicons, Aₙ or Bₙ, after n cycles, for two different targets (e.g., a wild-type target allele sequence and a rare mutant or rare variant target allele sequence), in a single reaction with limiting reagents (e.g., polymerase), calculated using the fraction of extendible complexes, “f.e.,” determined using the equations shown in FIG. 7. The present embodiments are based, in part, upon the recognition that the f.e. is preferably less than about 0.5, e.g. less than 0.4, 0.3, 0.2, 0.1, or less, for adequate blocking of amplification/detection of wild-type target allele sequences such that variant or mutant target allele sequences present in a sample at an initial copy number that is at 100-fold less (e.g., 200-fold, 300-fold, 400-fold, 500 fold, 600-fold, 700-fold, 800-fold, 900-fold, 1000-fold, 10000-fold or greater) than that of the wild-type target sequences.

Other considerations for design of the modulator oligonucleotide described herein are provided with reference to Diffenbach, C W et al., Genom. Res. S30-S37 (1993) and Rychik, W., Mol. Biotechnol. 3:129-134 (1995), each of which are incorporated herein by reference in its entirety. Although these references describe the design of amplification primers and the consideration for such designs, their discussion as to the need to consider free energy for hybridization of the 3′ terminus in order to control nonspecific hybridization to the native target and reduce the efficiency of target amplification is important for the design of modulator oligonucleotides.

### Internal Amplification Controls

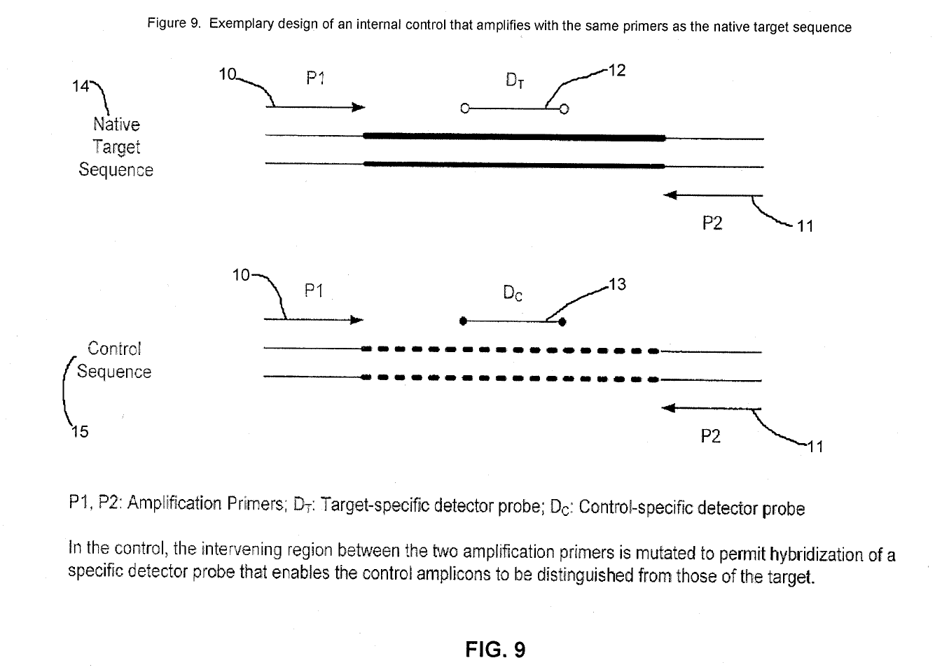

FIG. 9 depicts a typical design for an internal control where amplification of the native target sequence 14 and the internal amplification control 15 is accomplished with the same set of primers. For example, amplification primer P1 10 and amplification primer P2 11 are used to amplify regions of both the native target sequence 14 and the internal amplification control 15. The intervening region between the amplification primer P1 10 and amplification primer P2 11 in the internal amplification control 15 is mutated to permit the hybridization of a specific detector probe D_{C} 13 that enables the amplification products (amplicons) of the internal amplification control 15 to be distinguished from the amplicons of the native target sequence 14. A different detector probe D_{T} 12 is used to detect the presence of amplicons of the native target sequence. As discussed further herein, this design scheme often has many drawbacks, most problematic of which is the difference in amplification efficiencies between the native target and the internal amplification control.

The development of internal amplification controls typically involves an empirical process of trial and error to ensure that the chosen mutations to the target region and the associated control-specific detector probe do not significantly impair amplification of either the control itself or the target analyte. Several factors must be considered in the design of an appropriate internal amplification control. Such factors include, amplification efficiency, detection efficiency, discrimination, limit of detection (LOD), impact on native target LOD, robustness, and control effectiveness. For example, amplification efficiency is influenced by sequence length, GC content and the effects of secondary structure. The amplification efficiency of the internal control should approximate that of the target sequence.

It is important for the amplification efficiency of the internal control to be approximate to that of the target sequence. In order for that to be the case, primer sequence length, GC content and the effects of secondary structure on the primer must be taken into account. Similarly, detection efficiency of the internal control and target should be equivalent. This detection is influenced by, for example, the melting temperature of the probe, probe concentration and the presence of any secondary structure either in the probe or its complement. Furthermore, limits of detection for the control and native target sequence should be approximately equivalent. Thus, the internal control should not have an adverse impact in the analytical sensitivity of the assay for its specific target sequence.

It is also important that the internal control be readily distinguished from the target sequence and that there be no cross-reaction between the target and the control. The ability to discriminate the control from the target is influenced by the extent of the sequence differences between the two amplicons, as well as the specific design of the respective detector probes.

The internal amplification control should be robust to the anticipated variations in system parameters (e.g., temperature, chemical composition of the reaction buffer, etc.) that are within the design specifications for the assay system and not cause unnecessary reporting of unresolved or indeterminate results. The control should be present at a level that can be reproducibly delivered to the system, taking into account all manufacturing and system level variances such as, quantification of the stock, formulation of the control and the recovery of the control through the assay process. In order to prevent reporting of false-negative results, however, the response of the control to inhibitors or adverse conditions that fall outside those specified in the system design should mimic that of the target analyte.

Some of these may be inherently conflicting considerations that could require significant experimentation in order to obtain an acceptable balance in performance between amplification and detection of the control and of the target. For example, the need to ensure adequate discrimination between the target and the control dictates that the two sequences are sufficiently different to enable specific hybridization of different oligonucleotide probes and this requirement conflicts with the call for equivalence of amplification efficiency between the two amplicons. Changes in sequence that are necessary to distinguish the control from the target can either enhance or reduce amplification efficiency and the optimization of the control sequence is therefore largely an empirical process.

In order to improve the design and implementation of internal amplification controls that do not adversely impact the analytical or clinical sensitivity of the assay for the target analyte, the modulator oligonucleotides described herein can be used to suppress amplification of the control in favor of the target.

As discussed herein, modulator oligonucleotides are non-extendible oligonucleotides that are designed to hybridize to a region of the control sequence that at least partially overlaps the hybridization region of one of the amplification primers. The extent of overlap between the modulator oligonucleotide and the hybridization region of one of the primers is not critical and the number of nucleotides in the hybridization region that are hybridized to the modulator oligonucleotide is largely a matter of design choice. However, in order to avoid having an adverse impact on amplification of the target sequence, it is important that the modulator oligonucleotide hybridizes specifically to at least a portion of the primer binding region of the target sequence and control sequence. In addition, the extent of the overlap with the upstream amplification primer hybridization region, for example, should not be so great as to allow for stable hybridization of the 5′ end of the modulator oligonucleotide to its complement in the upstream amplification primer hybridization region of the control sequence without modulator oligonucleotide hybridization downstream of the primer hybridization region. For amplification reactions that deploy a pair of extendible primers to amplify the native target(s) and a control sequence, only one modulator oligonucleotide may be required to suppress amplification of the control sequence, in some embodiments. In another embodiment, a pair of modulator oligonucleotides is used.

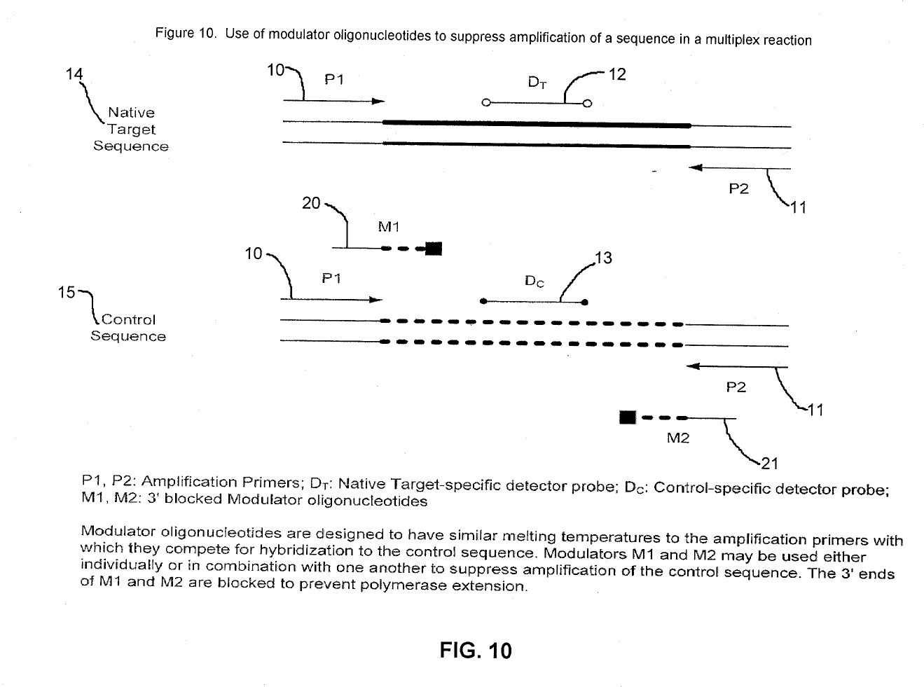

FIG. 10 depicts how modulator oligonucleotides may affect amplification efficiency. For example, modulator oligonucleotide M1 20 and modulator oligonucleotide M2 21 compete with the amplification primer P1 10 and amplification primer P2 11, respectively, for specific hybridization to the internal amplification control sequence. Competition between the amplification primers and modulator oligonucleotides reduces the amplification efficiency of the control sequence 15 relative to that of the specific target sequence 14.

In order to accomplish the reduced amplification efficiency, modulator oligonucleotide M1 20 is designed to have a nucleotide sequence that hybridizes to a portion of the control nucleotide sequence that overlaps at least a portion of the target binding region of the amplification primer P1 10 and the intervening region between the amplification primers P1 10 and P2 11. The intervening region is that portion of the internal amplification control that has a nucleotide sequence that is different from the nucleotide sequence of the native target. It is the nucleotide sequence of this intervening region that is used to distinguish the internal amplification control 15 from the native target 14. The target binding region of the modulator oligonucleotide on the internal amplification control is referred to as the overlap target binding region or the modulator oligonucleotide target region hereinafter.

Since the nucleotide sequence of the modulator oligonucleotide is such that it hybridizes to the overlap target binding region, the modulator oligonucleotide possesses adequate specificity for the internal amplification control such that only the amplification efficiency of the amplification control is affected by the presence of the modulator oligonucleotide. The amplification efficiency of the primers to the native target sequence is not significantly affected by the presence of the modulator oligonucleotide.

If desired, a second modulator oligonucleotide M2 21 may be used in addition to the first modulator (or instead of the first modulator) to further affect the amplification efficiency of the internal amplification control. Like the first modulator oligonucleotide M1 20, the second modulator oligonucleotide M2 21 is composed of a nucleotide sequence that targets an overlap target binding region of the internal amplification control. This overlap target region is a portion of the target binding region for amplification primer P2 11 and a portion of the control-specific nucleotide sequence between amplification primers P1 10 and P2 11. The above-described modulator oligonucleotide(s) mitigates the adverse effects that amplification of the chosen control sequence can have on the amplification and detection of the native target sequence.

A native target-specific detector probe D_{T} 12 is used to detect the presence of the native target and to determine the amplification efficiency of the native target sequence 14, amplification efficiency may be informed by either D_{T} alone or some combination of both D_{T} and D_{c}. Likewise, a control-specific detector probe D_{C} 13 is used to detect the presence of the internal amplification control and to determine the amplification efficiency of the internal amplification control sequence. Amplification efficiency of the control may be determined by either Dc alone, or some combination of D_{T} and D_{c}.

Also, the modulator oligonucleotide enables the control to be detected even when the limit of detection for the control is so much lower than that of the target that the quantity of the control sequence would otherwise be too low to allow reproducible delivery to the reaction mixture. Specifically, the presence of the modulator oligonucleotide permits there to be a higher, more reproducible amount of the control sequence in the reaction mixture without adversely affecting amplification efficiency of the target sequence.

### Kits

Aspects of the disclosure also relate to kits containing the reagents and compositions to carry out the methods described herein. Such a kit can comprise a carrier being compartmentalized to receive in close confinement therein one or more containers, such as tubes or vials. One of the containers may contain at least one unlabeled or detectably labeled primer or probe disclosed herein. The primers, including amplification primers, oligonucleotide modulators and detector probes can be present in dried form (e.g., lyophilized or other) or in an appropriate buffer as necessary. One or more containers may contain one or more enzymes or reagents to be utilized in PCR reactions. These enzymes may be present by themselves or in admixtures, in dried form or in appropriate buffers.

The kit may also include all of the additional elements necessary to carry out the methods disclosed herein, such as buffers, extraction reagents, enzymes, pipettes, plates, nucleic acids, nucleoside triphosphates, filter paper, gel materials, transfer materials, autoradiography supplies, and the like.

The kits according to embodiments disclosed herein may comprise at least: (a) a modulator oligonucleotide, (b) a forward and reverse amplification primer, (c) one or more sequence specific detector probe(s), e.g. allele specific probes, preferably detectably labeled, and (d) optionally instructions for using the provided amplification primer pair, modulator oligonucleotide, and probe(s).

In some embodiments, the kits include additional reagents that are required for or convenient and/or desirable to include in the reaction mixture prepared during the methods disclosed herein, where such reagents include: one or more polymerases; an aqueous buffer medium (either prepared or present in its constituent components, where one or more of the components may be premixed or all of the components may be separate), and the like. The various reagent components of the kits may be present in separate containers, or may all be pre-combined into a reagent mixture for combination with template nucleic acid.

In addition to the above components, in some embodiments, the kits can also include instructions for practicing the methods disclosed herein. These instructions can be present in the kits in a variety of forms, one or more of which may be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address that may be used via the internet to access the information at a removed site.

### EXAMPLES

The following examples are provided to demonstrate particular situations and settings in which this technology may be applied and are not intended to restrict the scope of the invention and the claims included in this disclosure.

### Example 1

The following example demonstrates that the methods disclosed herein can be used to effectively detect multiple rare variant target allele sequences in samples comprising an excess (100 fold or more) of wild-type or alternative variant or mutant target allele sequences.

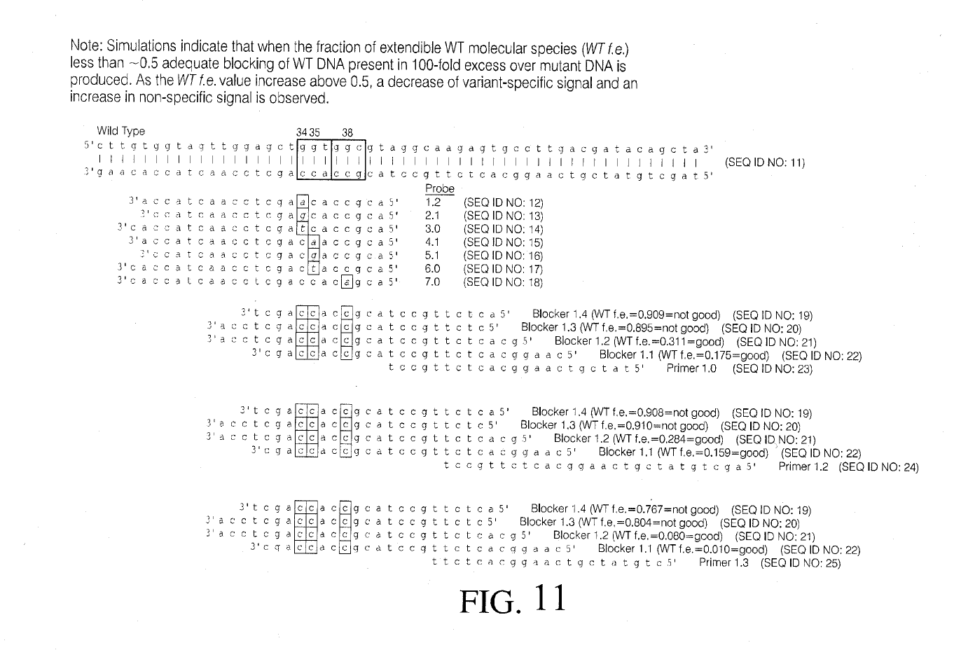

KRAS allelic variants G34T, G34C, G34A, and G38A, which are commonly used in the diagnosis prognosis of various cancers, as well as predicting the sensitivity of tumors to certain therapeutics, were used as an exemplary system to demonstrate the efficacy of the methods described herein. FIG. 11 shows the target region of interest in KRAS, including the wild-type sequence, as well as the position of the G34A, G34T and G38A variants.

Shown in FIG. 11 are three different amplification primers, i.e., Primer 1.0, Primer 1.2 and Primer 1.3 designed to amplify the target region of interest. Also shown are four different modulator/blocker oligonucleotides, i.e., blocker oligonucleotide 1.4, blocker oligonucleotide 1.3, blocker oligonucleotide 1.2 and blocker oligonucleotide 1.1 that include non-extendible 3′-OH modifications in accordance with the methods described above, and that are designed to preferentially binding to the wild-type target allele sequence compared to the various mutant allele sequences present at positions 34, 35, and 38 of KRAS, as shown in FIG. 11. Also shown are seven different detector probes, i.e., probes 1.2, 2.1, 3.0, 4.1, 5.1, 6.0 and 7.0 designed for the detection of G34A, wt, G34T, G35A, G35G, G35T and G38A alleles. The detector probes are configured to generate a detectable, fluorescent signal upon hybridization to target, measurable in real time.

Using the methods described herein above, the entropy, enthalpy, equilibrium constants, and fraction of each molecular species present at equilibrium were calculated as shown in FIGS. 6 and 7. These values were calculated for both wild-type and G34T DNA. Among the values calculated are the fraction of extendible molecular species (f.e.) wild-type (WT f.e.) and G34T (mutant f.e.) DNA. The calculated values also include the fraction of analyte (either wild-type or G34T) bound to extendible species containing a detector probe(s) (represented by PADE in FIGS. 6 and 7). The PADE species produce target amplification and detectable signal during PCR, whereas the other extendible species (PAE and PAbE) produce amplification but not detectable signal. For reaction mixtures containing more than one detector probe, the fraction of analyte involved in each PADE species was calculated, and these values are used to estimate the signal produced by each respective probe. The various f.e. values were used to perform PCR simulations in which the samples contained a 100-fold excess of wild-type target allele sequence compared to the G34T mutant allele sequence.

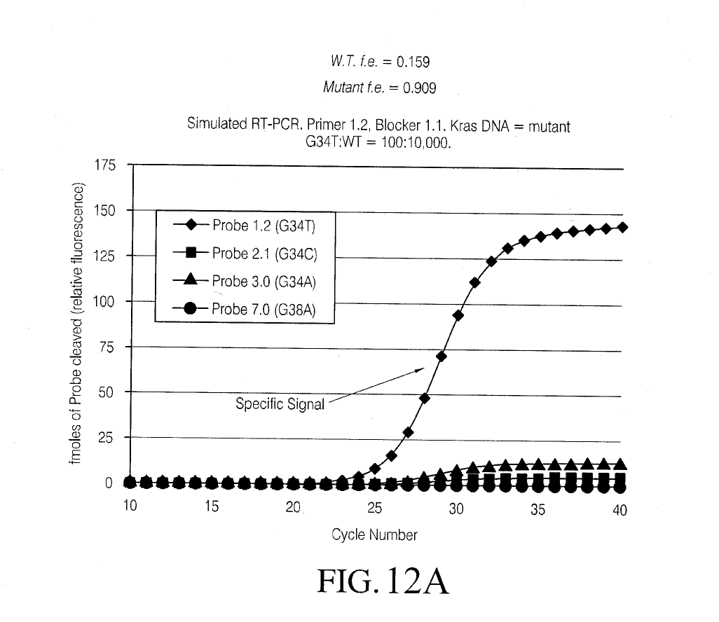

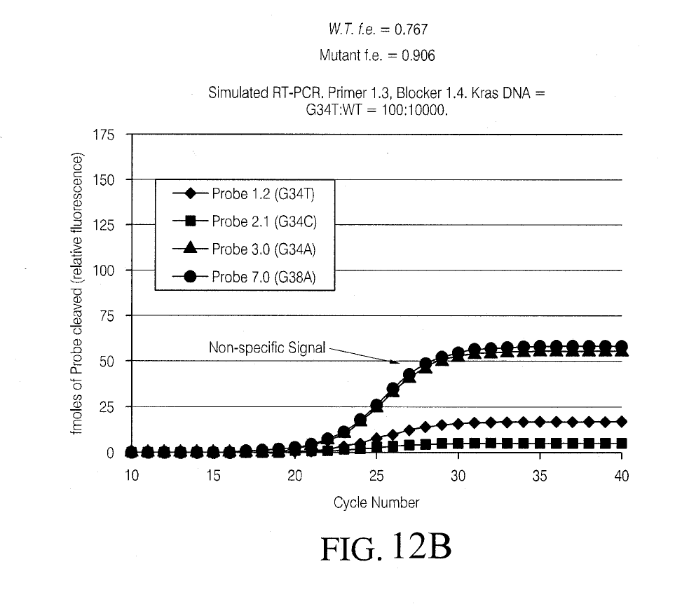

FIG. 12A shows the results of a simulated PCR reaction containing primer 1.2, blocker 1.1, and detector probes 1.2, 2.1, 3.0 and 7.0. As shown, a specific signal is detectable for G34T, whereas either very weak or no signal is produced from probes directed to the mutant target alleles not present in the sample. For this reaction, WT f.e. was 0.159 and mutant f.e. was 0.909, predictive of suppression of wild-type target amplification, but strong amplification of mutant target. In contrast, FIG. 12B shows the results of PCR simulation for reaction mixtures containing the same detector probes (1.2, 2.1, 3.0 and 7.0), but a different primer (primer 1.3) and blocker (blocker 1.4). Again, the wild-type allele is present in 100-fold excess over the mutant G34T allele. This primer-blocker combination results in calculated values for mutant f.e. of 0.906, and WT f.e. of 0.767, the latter of which is predictive of significant amplification of both wild-type and mutant target alleles. As shown in FIG. 12B, only weak signal is produced for the probe directed at the G34T allele, while significantly stronger signals are produced from probes directed at mutant alleles not present in the reaction mixture. These non-specific signals are produced by hybridization of probes to wild-type DNA, which because of the insufficient suppression of amplification by the blocker 1.4, is present at much higher levels than the G34T allele throughout the course of the PCR reaction.

The foregoing data demonstrate that the methods disclosed herein can be used to effectively detect and identify rare mutant or variant target allele sequences against a background of excess wild-type sequences. The methods disclosed herein thus represent an extremely efficient, efficacious means to detect sequence polymorphisms and mutations that have wide-ranging clinical and experimental uses, as well as more general application in multiplex reactions where one or more sequences are relatively rare or less abundant.

### Example 2

The following example demonstrates how the methods disclosed herein can be used to detect methyl cytosine residues in the death associated protein-1 (DAPK-1) promoter region. Changes in methylation status within the promoter region of DAKP-1 are frequently associated in with a variety of types of cancer and therefore accurate assessment of methylation patterns can be an important diagnostic indicator (Raval et al., (2007), Cell, 129: 879-890; Candiloro et al Epigenetics 2011 6: 500-507).

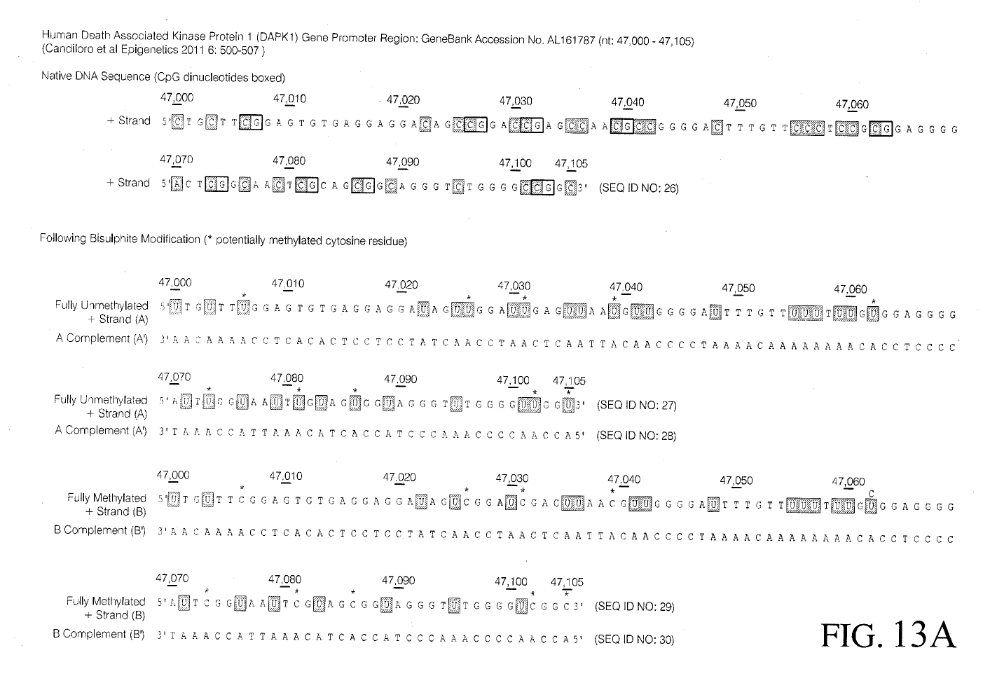

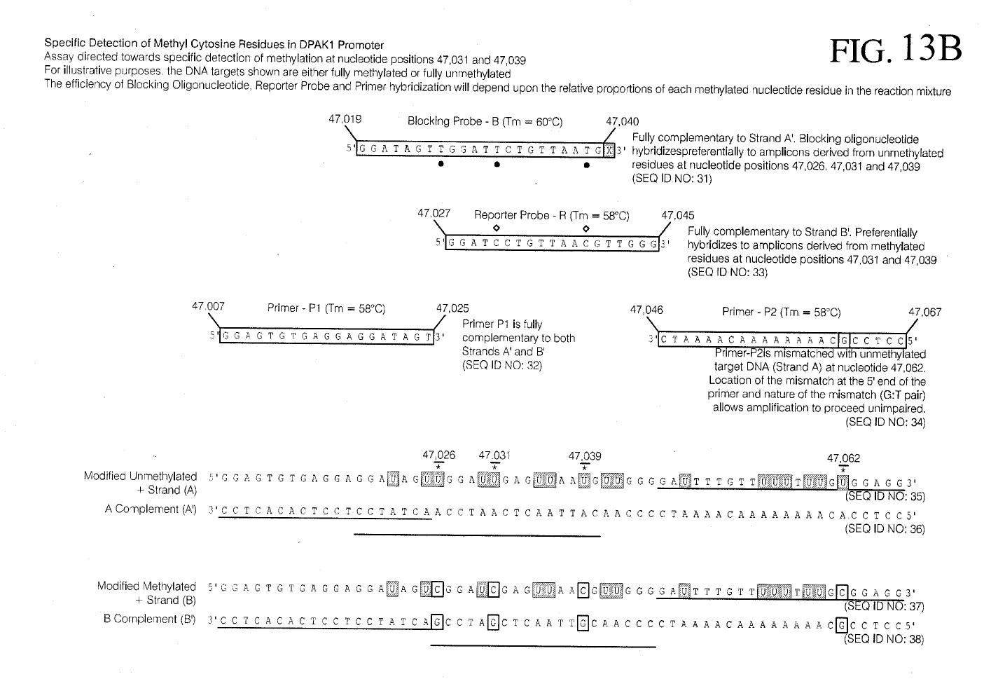

FIG. 13A shows a 105 bp target sequence within the promoter region of DAKP-1. CpG sites, which are often the sites of altered cytosine methylation patters, are shown in boxes. FIG. 13A also shows the unique sequences generated following treatment of the DAKP-1 promoter target sequence, when the sample DNA is originally fully unmethylated, or fully methylated. Specifically, as shown, there are nine cytosine residues that are potentially methylated, and that would be resistant to bisulphite treatment.

FIG. 13B shows a shorter, 61 bp region within the target sequence shown in FIG. 13A. As shown by the asterisks, four potential methylation sites, e.g., at nucleotide positions 47026, 47031, 47039 and 47062 exist within this region. Table 1 below illustrates the 16 possible DNA methylation patters within the DAPK-1 promoter region shown in FIG. 13B.

TABLE 1

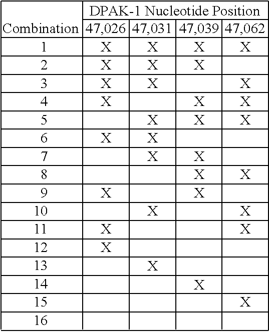

X: methyl cytosine residue;
Shaded box corresponds to residues detected by Reporter Probe-R

FIG. 13B illustrates how the use of methylation-specific amplification primers, methylation-specific reporter probes, and methylation-specific modulator oligonucleotides can be used to determine whether a sample comprising the DAPK-1 promoter target sequence comprises aberrant methylation. Primer P1 is fully complementary to sample DNA that is either fully methylated or unmethylated following modification with sodium bisulphite. By contrast, primer P2 includes a guanine residue that is mismatched with a converted uracil residue in the modified sample nucleic acids from a fully unmethylated sample, but which is complementary to modified sample nucleic acids from a fully methylated sample. Due to the fact that the mismatch is not at the 3′ end of the reverse primer, however, amplification can still occur under standard amplification conditions. The reporter probe R contains 2 cytosine residues that are mismatched with the modified sample nucleic acids from a fully unmethylated sample, but which are complementary to modified sample nucleic acids from a fully methylated sample. As such, the reporter probe preferentially hybridizes to the amplicon derived from sample nucleic acids that are methylated, compared to amplicons derived from sample nucleic acids that are unmethylated. Modulator/blocker oligonucleotide (a.k.a “blocking probe”) B includes 3 thymine residues that hybridize to uracil residues present in the modified unmethylated sample, but that are mismatched with the guanine residues present in the modified methylated sample. The modulator/blocker oligonucleotide contains a modification at its 3′ end that inhibits extension. As such, the modulator/blocker oligonucleotide will preferentially hybridize to amplicons derived from the unmethylated sample nucleic acids, as compared to the methylated sample nucleic acids. Accordingly, using primers P1, P2, reporter probe R, and modulator/blocker oligonucleotide B, one can preferentially amplify and detect rare methylated sample nucleic acids, e.g., within a sample comprising an abundance of unmethylated nucleic acids.

### Example 3

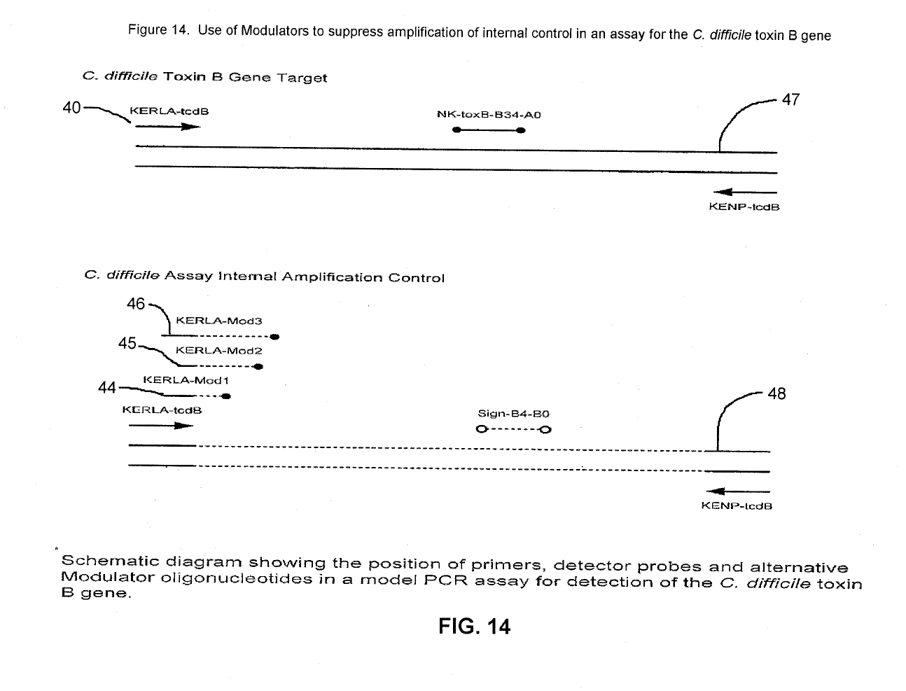

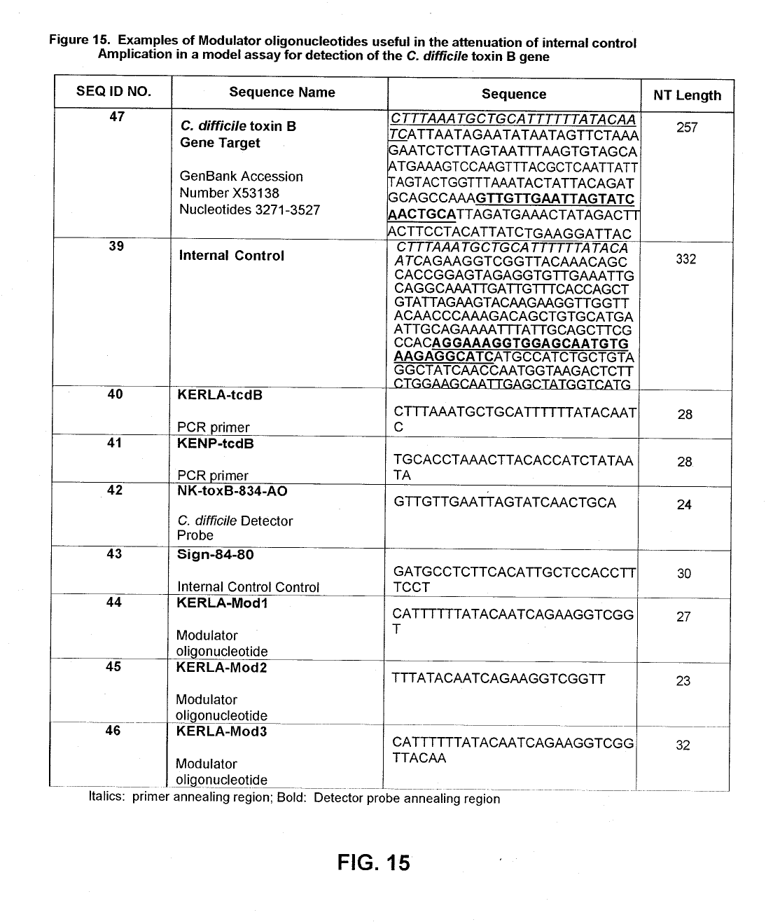

FIG. 14 illustrates the relative target binding positions of the two primers, KERLA-tcdB 40 and KENP-tcdB 41, two probes, NK-toxB-B34-AD 42 and Sign-B4-B0 43, and three modulator oligonucleotides, KERLA-Mod1 44, KERLA-Mod2 45, and KERLA-Mod3 46, relative to the Clostridium difficile toxin B gene target 47 and internal control sequence 48. The three modulator oligonucleotides, KERLA-Mod1 44, KERLA-Mod2 45, and KERLA-Mod3 46, differ with respect to their melting temperatures (relative to the target) and the degree of overlap with the target binding region of the upstream amplification primer KERLA-tcdB 40 to the Clostridium difficile toxin B gene target. The sequences for these three alternative modulators are listed in FIG. 15, and their characteristics are listed in Table 2. The modulator oligonucleotides described in Table 2 attenuate amplification of the internal control sequence of a model PCR-based assay for detection of the toxin B gene of C. difficile. Table 2 lists the length, Tₘ, and extent of overlap between the modulator oligonucleotides, i.e., KERLA-Mod1 44, KERLA-Mod2 45, and KERLA-Mod3 46, and the upstream amplification primer KERLA-tcdB 40.

TABLE 2
Comparison of the characteristics of the competing modulator and
primer oligonucleotides in the C. difficile model assay system

| | |
|---|---|
| Melting | |
| Temperature | Overlap with |
| (° C.) | KERLA-tcdB |

| Name | Length | Td* | Tm** | (nucleotides) |
|---|---|---|---|---|
| KERLA-tcdB | 28 | 68.2 | 70 | |
| KERLA-Mod1 | 27 | 69.5 | 72 | 16 |
| KERLA-Mod2 | 23 | 63.1 | 62 | 11 |
| KERLA-Mod3 | 32 | 74.7 | 84 | 16 |

*Nearest neighbor
**Wallace-Ikatura Rule (von Ahsen et al Clin Chem 2001 47: 1956-1961)

The preferred concentration of the modulator oligonucleotide may be determined empirically, or as described herein, and is influenced by the Tₘ of the modulator oligonucleotide relative to that of the amplification primer with which it competes for hybridization. The higher the Tₘ of the modulator oligonucleotide relative to that of the competing primer, the lower the concentration of modulator oligonucleotide required to suppress amplification of the control.

Table 3 describes theoretical calculations of the fraction of Internal Control bound to the amplification primer KERLA-tcdB 40, shown in FIG. 14 and listed in FIG. 15, in the presence of various concentrations of the three modulator oligonucleotides, KERLA-Mod1 44, KERLA-Mod2 45, and KERLA-Mod3 46, also shown in FIG. 14 and listed in FIG. 15. Calculations were based on a three-state equilibrium hybridization model in which the following three hybridization scenarios may occurs: (1) neither the primer, nor modulator are hybridized to the control sequence (2) primer is hybridized to control sequence (3) modulator is hybridized to control sequence. In this example the reactions may be conducted at a temperature of 55° C. with the primer concentration set at 200 nM and a sodium ion concentration of 150 mM. Under these conditions, in the absence of any modulator oligonucleotide (modulator oligonucleotide concentration equal to zero 0.0; Table 3), the fraction of Internal Control molecules hybridized to primer at 55° C. is calculated to be 0.96 (or 96%).

TABLE 3
Theoretically calculated fraction of Internal Control hybridized
to primer (KERLA-tcdB) at 55° C. in the presence of
modulators at varying concentrations*

Modulator Concentration (μM)

| Modulator | 0.0 | 0.01 | 0.1 | 1.0 |
|---|---|---|---|---|
| KERLA-Mod1 | 0.96 | 0.48 | 0.086 | 0.009 |
| KERLA-Mod2 | 0.96 | 0.92 | 0.69 | 0.19 |
| KERLA-Mod3 | 0.96 | 0.052 | 0.005 | 0.0005 |

*Calculations determined from three-state hybridization model with KERLA-tcdB concentration of 0.2 μM, and Na⁺ concentration of 150 mM.

Referring to Table 3, if any modulator oligonucleotide is present, the fraction of Internal Control bound to the primer is reduced. As noted above, the amount of primer-bound Internal Control decreases as modulator oligonucleotide concentration increases. A reduction in the amount of Internal Control bound to primer is expected to modulate the amplification efficiency of Internal Control. Table 3 also illustrates a correlation between the lengths and Tₘ of the modulator oligonucleotides and the degree of primer-Internal Control hybridization suppression. Longer modulator oligonucleotides, with higher Tₘ, suppress primer hybridization to Internal Control more efficiently.

In contrast, as shown in Table 4, the presence of modulator oligonucleotides KERLA-Mod1 44, KERLA-Mod2 45, or KERLA-Mod3 46 do not detectably affect the fraction of C. difficile target hybridized to primer over the 100-fold range of modulator oligonucleotide concentrations examined in these calculations. Thus, the presence of modulator oligonucleotides KERLA-Mod1 44, KERLA-Mod2 45, or KERLA-Mod3 46 is not expected to affect the amplification efficiency of the C. difficile Toxin B gene target significantly.

TABLE 4
Theoretically calculated fraction of C. difficile toxin B Gene
Target hybridized to primer (KERLA-tcdB) at 55° C. in the
presence of modulators at various concentrations*

Modulator Concentration (μM)

| Modulator | 0.0 | 0.01 | 0.1 | 1.0 |
|---|---|---|---|---|
| KERLA-Mod1 | 0.96 | 0.96 | 0.96 | 0.96 |
| KERLA-Mod2 | 0.96 | 0.96 | 0.96 | 0.96 |
| KERLA-Mod3 | 0.96 | 0.96 | 0.96 | 0.96 |

*Calculations determined from three-state hybridization model with KERLA-tcdB concentration of 0.2 μM, and Na⁺ concentration of 150 mM.

As illustrated in the example provided herein, selecting a modulator oligonucleotide of a certain predetermined length and controlling its concentration, the amplification efficiency of the Internal Control is tuned to match the amplification efficiency of the target DNA.

The embodiments described and claimed herein is not to be limited in scope by the specific embodiments herein disclosed, since these embodiments are intended as illustrations of several aspects of the invention. Any equivalent embodiments are intended within the scope of this invention. Indeed, various modifications of the embodiments in addition to those shown and described herein will become apparent to those skilled in the art from the foregoing description. The appended claims are intended to cover such modifications.

What is claimed is:

1. A method to modulate the amplification efficiency of a nucleic acid sequence in an amplification reaction, the method comprising:
providing an amplification reaction comprising a pair of amplification primers comprising a forward primer and a reverse primer, said pair of primers configured to amplify a first target nucleic acid and thereby produce a first amplicon having a first nucleic acid sequence, and configured to amplify a second target nucleic acid and thereby produce a second amplicon having a second nucleic acid sequence, wherein a portion of the second nucleic acid sequence is different from the first nucleic acid sequence;
providing a modulator oligonucleotide to the amplification reaction, wherein the modulator oligonucleotide preferentially hybridizes to the second nucleic acid sequence in comparison to the first nucleic acid, and wherein at least a portion of the modulator oligonucleotide shares sequence identity to either the forward or reverse primer, and the remainder of the modulator oligonucleotide hybridizes to at least a portion of the second nucleic acid sequence that differs from the first nucleic acid sequence;
amplifying the nucleic acid sequences, wherein the modulator oligonucleotide reduces the amplification efficiency of the second amplicon by competing with at least one of the forward or reverse primer for binding to the second target nucleic acid.

2. The method of claim 1, wherein the method further comprises providing a first reporter probe specific for the first amplicon and a second reporter probe specific for the second amplicon.

3. The method of any of the preceding claims wherein the first target nucleic acid is less abundant than the second nucleic acid.

4. The method of any of the preceding claims wherein the first target nucleic acid is a rare nucleic acid.

5. The method of any of the preceding claims wherein the first target nucleic acid contains an allelic variation and the second target nucleic acid is a wild-type nucleic acid.

6. The method of any of claims 1-3, wherein the second target nucleic acid is an internal control nucleic acid.

7. The method of any of the preceding claims further comprising detecting the presence of the first amplicon and/or the second amplicon using a first reporter probe specific for the first amplicon and/or a second reporter probe specific for the second amplicon.

8. A method to detect a first variant target sequence in a sample comprising nucleic acids, the method comprising:
providing the sample;
contacting the sample with:
a pair of amplification primers comprising a forward primer and a reverse primer, said pair of amplification primers configured to amplify a target amplicon, wherein said amplicon comprises a wild-type target sequence or a variant target sequence, and wherein the pair of amplification primers amplifies both wild-type target sequences and variant target sequences;
a modulator oligonucleotide that preferentially hybridizes to the wild type target sequence compared to a first variant target sequence under amplification conditions; and
a reporter probe, wherein said reporter probe comprises an oligonucleotide that preferentially hybridizes to the first variant target sequence compared to the wild-type target sequence under amplification conditions; wherein said contacting takes place under amplification conditions; and
measuring the hybridization of the reporter probe to the first variant target sequence, wherein hybridization of the reporter probe to the first variant target sequence produces a detectable signal indicative of the presence or amount of first variant target species in the sample.

9. The method of any of the preceding claims, wherein the amplification mixture comprises extendible molecular species of target amplicons and non-extendible molecular species of target amplicons, and wherein a fraction of extendible species (f.e.) represents the fraction of extendible species of a total number target amplicons.

10. The method of any of the preceding claims, wherein the f.e. is less than about 0.5.

11. The method of any of the preceding claims, wherein the sample comprises about 100-fold excess of wild-type target sequences compared to variant target sequence.

12. The method of any of the preceding claims, further comprising detecting a second variant target sequence, wherein the modulator oligonucleotide preferentially hybridizes to the wild type target sequence compared to the second variant target sequence under amplification conditions, wherein said method further comprises:
contacting the sample with a second reporter probe, wherein said second reporter probe comprises an oligonucleotide that preferentially hybridizes to the second variant target sequence compared to the wild-type target sequence under amplification conditions; wherein said contacting takes place under amplification conditions; and
measuring the hybridization of the second reporter probe to the second variant target sequence, wherein hybridization of the reporter probe to the second variant target sequence produces a detectable signal indicative of the presence or amount of second variant target species in the sample.

13. The method of the preceding claims, wherein the sample is simultaneously contacted with the first reporter probe and the second reporter probe.

14. The method of any of the preceding claims, wherein the first and/or second reporter probe comprises a modified nucleic acid.

15. The method of any of the preceding claims, wherein the first variant target sequence is in a gene selected from the group consisting of: KRAS, BRAF, EGFR, TP53, JAK2, NPM1, and PCA3.

16. The method of any of the preceding claims, wherein the second variant target sequence is in a gene selected from the group consisting of: KRAS, BRAF, EGFR, TP53, JAK2, NPM1, and PCA3.

17. The method of any of the preceding claims, wherein the method comprises performing real-time PCR.

18. The method of any of the preceding claims, wherein the method comprises performing isothermal amplification.

19. A method for modulating the amplification of a nucleic acid sequence comprising:
(a) providing a reaction mixture comprising a sample suspected to contain a target nucleic acid and a primer capable of hybridizing to the target nucleic acid under conditions that will cause at least some of the primer to hybridize to the target nucleic acid if present, wherein the reaction mixture further comprising a modulator oligonucleotide capable of selectively hybridizing to a control nucleic acid, wherein the reaction mixture is subjected to conditions that will cause the modulator oligonucleotide to hybridize to the control nucleic acid;
(b) subjecting the reaction mixture to conditions for amplifying the target nucleic acid, if present, and the control nucleic acid, wherein the amplification conditions permit the primer and modulator oligonucleotide to hybridize to the control nucleic acid at similar melting temperatures, wherein the reaction mixture further comprises a first reporter probe specific for the target nucleic acid and a second reporter probe specific for the control nucleic acid; and
(c) subjecting the reaction mixture to conditions under which the first reporter probe hybridizes to the target nucleic acid, if present, and the second reporter probe hybridizes to the control nucleic acid wherein the reaction mixture is monitored to detect the hybridization of the respective probes to their respective targets.

20. A method of detecting the presence of a methylated cytosine residue in a target DNA sequence in a sample, comprising:
treating the sample with a reagent that specifically modifies unmethylated cytosine residues to uracil residues to generate a modified sample DNA to generate a modified sample DNA target sequence;
combining the modified sample DNA target sequence with an amplification primer pair comprising a forward primer and a reverse primer, wherein the forward and reverse amplification primers are fully complementary to modified sample DNA that comprises methylated cytosines, and that is not fully complementary to modified sample DNA that comprises uracil residues to create an amplification reaction mixture;
contacting the reaction mixture with a reporter probe that is fully complementary to target amplicons generated from modified sample DNA that comprises methylated cytosines, and that is not fully complementary to target amplicons generated from modified sample DNA that comprises uracil;
subjecting the reaction mixture to an amplification reaction to generate target amplicons;
detecting the amount of reporter probe bound to target amplicons produced from the amplification reaction.

21. The method of claim 20, wherein the reaction mixture further comprises a modulator oligonucleotide that competes with the reverse primer and/or the reporter probe for hybridizing to the amplified target sequence, wherein the modulator oligonucleotide preferentially hybridizes to amplicons produced from modified sample DNA that comprises uracil residues.

22. The method of any of the preceding claims, wherein the modulator oligonucleotide is between 15 and 30 nucleotides in length.

23. The method of any of the preceding claims, wherein the first and/or second reporter probe is between 15 and 30 nucleotides in length.

24. The method of any of the preceding claims, wherein the modulator oligonucleotide is longer than the first and/or second reporter probe.

25. The method of any of the preceding claims, wherein the first and/or second reporter probe does not overlap with either the forward or reverse amplification primer.

26. The method of any of the preceding claims, wherein the first and/or second reporter probe overlaps with the modulator oligonucleotide, wherein the overlap between the first and/or second reporter probe and the modulator oligonucleotide does not extend to the 3′ end of the reporter probe.

27. The method of any of the preceding claims, wherein the first and/or second reporter probe overlaps with the modulator oligonucleotide, wherein the overlap between the first and/or second reporter probe and the modulator oligonucleotide does not extend to the 5′ end of the modulator oligonucleotide.

28. The method of any of the preceding claims, wherein the modulator oligonucleotide overlaps with either the forward or reverse amplification primer, and wherein the overlap does not extend to the 3′ end of the modulator oligonucleotide.

29. The method of claim 28, wherein the overlap between the modulator oligonucleotide and the forward or reverse amplification primer does not extend to the 5′ end of the forward or reverse amplification primer.

30. The method of any of the preceding claims, wherein the first reporter probe and/or second is selected from the group consisting of a TAQMAN® reporter probe, a SCORPION® reporter probe, a hybridization (FRET) probe, and a molecular beacon probe.
